(19) United States
(12) Patent Application Publication
(10) Pub. No.: US 2022/0146434 A1
(43) Pub. Date: May 12, 2022

(54) IMAGE PROCESSING APPARATUS, INFORMATION GENERATION APPARATUS, AND METHOD THEREOF

(71) Applicant: SONY GROUP CORPORATION, TOKYO (JP)

(72) Inventors: LEGONG SUN, TOKYO (JP); YASUTAKA HIRASAWA, KANAGAWA (JP); YUHI KONDO, TOKYO (JP); TAISHI ONO, (US)

(21) Appl. No.: 17/593,614

(22) PCT Filed: Jan. 15, 2020
(86) PCT No.: PCT/JP2020/000994

§ 371 Date: Sep. 21, 2021

(30) Foreign Application Priority Data
Apr. 3, 2019 (JP) 2019-071117

(51) Int. Cl. G01N 21/88; G01N 21/21

(52) U.S. Cl. CPC G01N 21/8806; G01N 2021/8848; G01N 21/21

(57) ABSTRACT

A determination environment information acquisition unit 31 acquires incident polarized light information of a light source in a material determination environment. A determination target information acquisition unit 32 acquires emitted polarized light information from a polarized image obtained by capturing an image of a material determination target in the material determination environment. A determination processing unit 34 can determine a material of the material determination target on the basis of the incident polarized light information acquired at the determination environment information acquisition unit 31, the emitted polarized light information acquired at the determination target information acquisition unit 32, and material polarizing characteristic information which is stored in advance in an information storage unit 33, that is, material polarizing characteristic information which indicates, for each material, polarizing and reflecting characteristics for each incident direction of incident polarized light and for each emission direction of reflected light.

### TECHNICAL FIELD

The present technology relates to an image processing apparatus, an information generation apparatus, and a method thereof, which enable easy determination of a material on the basis of a polarized image.

### BACKGROUND ART

As a determination method of a material in related art, Patent Document 1 discloses specifying a type of an unknown material by capturing light which has passed through the material or which has been reflected from the material, dispersing the light, and comparing signal information generated on the basis of the dispersed light and signal information of materials registered in advance.

### CITATION LIST

### Patent Document

Patent Document 1: Japanese Patent Application Laid-Open No. 2002-243639

### SUMMARY OF THE INVENTION

### Problems to be Solved by the Invention

By the way, signal information of materials registered in advance is measurement results under a certain environment and conditions. Thus, it is necessary to provide signal information of materials for each environment and for each condition in advance to specify a type of a material on the basis of signal information generated on the basis of dispersed light. Further, it is necessary to measure an environment or a condition every time the environment or the condition changes in a case where the environment or the condition changes, as well as generate signal information, and thus, the type of the material cannot be easily determined.

The present technology is therefore directed to providing an image processing apparatus, an information generation apparatus, and a method thereof, which enable easy determination of a material on the basis of a polarized image.

### Solutions to Problems

According to a first aspect of the present technology,

there is provided an image processing apparatus including:

a determination environment information acquisition unit configured to acquire incident polarized light information of a light source in a material determination environment;

a determination target information acquisition unit configured to acquire emitted polarized light information from a polarized image obtained by capturing an image of a material determination target in the material determination environment; and

a determination processing unit configured to determine a material of the material determination target on the basis of the incident polarized light information acquired at the determination environment information acquisition unit, the emitted polarized light information acquired at the determination target information acquisition unit and material polarizing characteristic information which indicates polarizing and reflecting characteristics for each incident direction of incident polarized light and for each emission direction of reflected light, and which is generated in advance.

In this technology, the determination environment information acquisition unit acquires incident polarized light information, for example, an incident Stokes vector of a light source in the material determination environment. Further, the determination target information acquisition unit acquires emitted polarized light information, for example, an emitted Stokes vector from a polarized image obtained by capturing an image of the material determination target in the material determination environment. The determination processing unit uses incident polarized light information, emitted polarized light information, and material polarizing characteristic information which indicates a Mueller matrix and which is generated in advance and stored in an information storage unit as polarizing and reflecting characteristics for each of incident direction of the incident polarized light and for each emission direction of reflected light. Further, the determination processing unit may use normalized incident polarized light information, emitted polarized light information and material polarizing characteristic information.

The determination processing unit calculates an error of one of the incident polarized light information and the emitted polarized light information estimated using the material polarizing characteristic information selected in accordance with the incident direction of the incident polarized light on the material determination target and the emission direction of the reflected light from the material determination target and the other of the incident polarized light information and the emitted polarized light information. For example, the determination processing unit generates estimated emitted polarized light information using the selected material polarizing characteristic information and the incident polarized light information and calculates an error between the estimated emitted polarized light information and the emitted polarized light information acquired at the determination target information acquisition unit. Further, the determination processing unit may calculate estimated incident polarized light information using the selected material polarizing characteristic information and the emitted polarized light information acquired at the determination target information acquisition unit and may calculate an error between the estimated incident polarized light information and the incident polarized light information acquired at the determination environment information acquisition unit.

The determination processing unit determines the material of the material determination target on the basis of the calculated error. For example, the material polarizing characteristic information is generated for each of a plurality of materials, and the determination processing unit determines a material with a minimum error as the material of the material determination target in a case where the minimum error among the calculated errors or the minimum error is smaller than a threshold set in advance. The determination environment information acquisition unit segments the material determination environment into a plurality of regions and sets an average incident direction and average incident polarized light information for each region as an incident direction and incident polarized light information of the region. Further, the determination environment information acquisition unit may acquire the incident polarized light information for each of a plurality of light sources in the material determination environment, and the determination processing unit may calculate an error using the incident polarized light information for each of the plurality of light sources and may calculate an error using incident polarized light information of a light source selected from the incident polarized light information for each of the plurality of light sources.

Further, the image processing apparatus may further include a detection region setting unit configured to set a target subject detection region from a polarized image obtained by capturing an image of the material determination target; and a region detection unit configured to detect a target subject region from the target subject detection region set at the detection region setting unit on the basis of a material determination result at the determination processing unit.

According to a second aspect of the present technology,

there is provided an image processing method including:

acquiring incident polarized light information of a light source in a material determination environment at a determination environment information acquisition unit;

acquiring emitted polarized light information from a polarized image obtained by capturing an image of a material determination target in the material determination environment at a determination target information acquisition unit; and

determining a material of the material determination target at a determination processing unit on the basis of the incident polarized light information acquired at the determination environment information acquisition unit, the emitted polarized light information acquired at the determination target information acquisition unit and material polarizing characteristic information which indicates, for each material, polarizing and reflecting characteristics for each incident direction of incident polarized light and for each emission direction of reflected light, and which is generated in advance.

According to a third aspect of the present technology,

there is provided an information generation apparatus including:

a light source information acquisition unit configured to acquire incident polarized light information of incident polarized light on an information generation target from a light source in a measurement environment in which the information generation target whose material is obvious is provided, for each incident direction;

an emitted polarized light information acquisition unit configured to acquire emitted polarized light information of reflected light from the information generation target for each emission direction; and

a material polarizing characteristic information generation unit configured to generate material polarizing characteristic information which indicates polarizing and reflecting characteristics in an incident direction of the incident polarized light and in an emission direction of the reflected light for each direction using the incident polarized light information acquired at the light source information acquisition unit and the emitted polarized light information acquired at the emitted polarized light information acquisition unit.

In the present technology, the light source information acquisition unit acquires the incident polarized light information, for example, an incident Stokes vector of incident polarized light on an information generation target from a light source in a measurement environment in which the information generation target whose material is obvious is provided, for each incident direction and for each material. The emitted polarized light information acquisition unit acquires the emitted polarized light information, for example, an emitted Stokes vector of reflected light from the information generation target for each emission direction and for each material on the basis of an information generation target imaging unit configured to generate polarized images in a plurality of polarization directions by capturing an image of the information generation target and observation values of polarized images generated at the information generation target imaging unit. The material polarizing characteristic information generation unit generates material polarizing characteristic information which indicates polarizing and reflecting characteristics in the incident direction of the incident polarized light and in the emission direction of the reflected light, for example, a Mueller matrix, for each direction and for each material using the incident polarized light information and the emitted polarized light information. Further, the material polarizing characteristic information generation unit may generate normalized material polarizing characteristic information.

According to a fourth aspect of the present technology

there is provided an information generation method including:

acquiring incident polarized light information of incident polarized light on an information generation target from a light source in a measurement environment in which the information generation target whose material is obvious is provided, for each incident direction at a light source information acquisition unit;

acquiring emitted polarized light information of reflected light from the information generation target for each emission direction at an emitted polarized light information acquisition unit; and

generating material polarizing characteristic information which indicates polarizing and reflecting characteristics in an incident direction of the incident polarized light and in an emission direction of the reflected light at a material polarizing characteristic information generation unit for each direction using the incident polarized light information acquired at the light source information acquisition unit and the emitted polarized light information acquired at the emitted polarized light information acquisition unit.

### BRIEF DESCRIPTION OF DRAWINGS

FIG. 1 is a view for explaining polarizing and reflecting characteristics.

FIG. 2 is a view illustrating an example of a configuration of a material determination system.

FIG. 3 is a view illustrating an example of a configuration of an information generation apparatus.

FIG. 4 is a flowchart illustrating an example of operation of the information generation apparatus.

FIG. 5 is a flowchart (No. 1) illustrating the operation of the information generation apparatus in detail.

FIG. 6 is a flowchart (No. 2) illustrating the operation of the information generation apparatus in detail.

FIG. 7 is a view illustrating an example of material polarizing characteristic information.

FIG. 8 is a view illustrating an example of a configuration of an image processing apparatus.

FIG. 9 is a view illustrating an example of a configuration of an environment imaging unit 311.

FIG. 10 is a view illustrating an example of a polarized image generated at the environment imaging unit 311.

FIG. 11 is a view illustrating an example of division of the polarized image.

FIG. 12 is a flowchart illustrating an example of operation of the image processing apparatus.

FIG. 13 is a flowchart illustrating a first operation example.

FIG. 14 is a flowchart illustrating a second operation example.

FIG. 15 is a flowchart illustrating a third operation example.

FIG. 16 is a view illustrating a third operation example.

FIG. 17 is a view illustrating an example of a case of distinguishing among objects which have substantially the same appearance.

FIG. 18 is a view illustrating an example of a case of presenting a determination result in pixel unit.

FIG. 19 is a view illustrating an example of other methods for acquiring polarized images.

### MODE FOR CARRYING OUT THE INVENTION

An embodiment for implementing the present technology will be described below. Note that description will be provided in the following order.

1. Polarizing and reflecting characteristics

2. Configuration of material determination system

3. Information generation apparatus

3-1. Configuration of information generation apparatus

3-2. Operation of information generation apparatus

4. Image processing apparatus

4-1. Configuration of image processing apparatus

4-2. Operation of image processing apparatus

4-2-1. First operation of determination processing unit

4-2-2. Second operation of determination processing unit

5. Operation example of image processing apparatus

5-1. First operation example

5-2. Second operation example

5-3. Third operation example

5-4. Other operation examples

6. Other configurations and operation

7. Application examples

### 1. Polarizing and Reflecting Characteristics

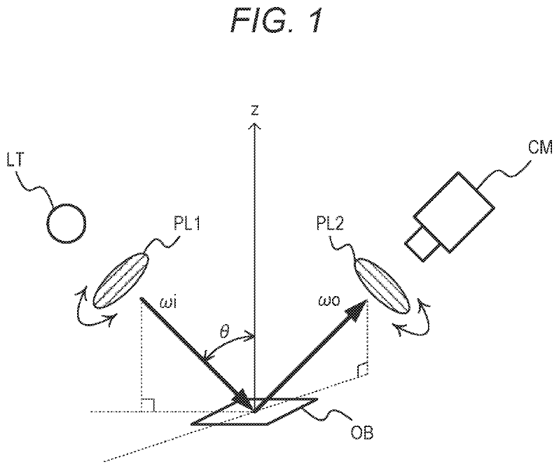

FIG. 1 is a view for explaining polarizing and reflecting characteristics. A measurement object OB is irradiated with light emitted from a light source LT via a polarizer, for example, a linear polarizer PL1, and an imaging apparatus CM captures an image of the measurement object OB, for example, via a linear polarizer PL2. Note that a Z direction indicates a zenith direction, and an angle θ is a zenith angle.

In a case where polarization directions of the linear polarizers (PL1 and PL2) are set at, for example, 0°, 45°, 90° and 135°, and a pixel value obtained by capturing an image of the measurement object with the imaging apparatus CM is set as an observation value I, relationship among an observation value I (0°) in a case where the polarization direction is 0°, an observation value I (45°) in a case where the polarization direction is 45°, an observation value I (90°) in a case where the polarization direction is 90°, and an observation value I) (135° in a case where the polarization direction is 135° can be indicated as a Stokes vector VS=[s⁰, s¹, s²]^{T}. Note that relationship between the Stokes vector and the observation values is as indicated with Expression (1).

$$\begin{matrix}[ Math . 1 ] & \\ VS = [ \begin{matrix}{S}^{0} \\ {S}^{1} \\ {S}^{2}\end{matrix} ] = [ \begin{matrix}I ( 0 ° ) + I ( 90 ° ) \\ I ( 0 ° ) - I ( 90 ° ) \\ I ( 45 ° ) - I ( 135 ° )\end{matrix} ] & ( 1 )\end{matrix}$$

In the Stokes vector, the component s⁰ indicates luminance of non-polarization or average luminance. Further, the component s¹ indicates a difference in strength in the polarization direction between 0° and 90°, and the component s² indicates a difference in strength in the polarization direction between 45° and 135°. In other words, the Stokes vector of 0° becomes [1, 1, 0]^{T}, the Stokes vector of 45° becomes [1, 0, 1]^{T}, the Stokes vector of 90° becomes [1, −1, 0]^{T}, and the Stokes vector of 135° becomes [1, 0, −1]^{T}.

Here, in a case where a Stokes vector of light in an incident direction ωi radiated on the measurement object OB is set as “VSi”, a Stokes vector of light in an emission direction coo measured at the imaging apparatus CM is set as “VSo”, and a Mueller matrix in a case of the incident direction ωi and the emission direction ωo is set as M(ωo, ωi), Expression (2) holds. Note that Expression (3) is determinant representation of Expression (2).

$$\begin{matrix}M ( ω o , ω i ) \cdot VSi = VSo & ( 2 ) \\ [ Math . 2 ] & \\ [ \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ] [ \begin{matrix}{S}_{i}^{0} \\ {S}_{i}^{1} \\ {S}_{i}^{2}\end{matrix} ] = [ \begin{matrix}{S}_{o}^{0} \\ {S}_{o}^{1} \\ {S}_{o}^{2}\end{matrix} ] & ( 3 )\end{matrix}$$

Expression (3) becomes Expression (4) in a case where the polarization direction of incident light radiated on the measurement object OB is 0°. Further, Expression (3) becomes Expression (5) in a case where the polarization direction of the incident light is 45°, Expression (3) becomes Expression (6) in a case where the polarization direction of the incident light is 90°, and Expression (3) becomes Expression (7) in a case where the polarization direction of the incident light is 135°.

$$\begin{matrix}[ Math . 3 ] & \\ [ \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ] [ \begin{matrix}1 \\ 1 \\ 0\end{matrix} ] = [ \begin{matrix}{S}_{0 °}^{0} \\ {S}_{0 °}^{1} \\ {S}_{0 °}^{2}\end{matrix} ] & ( 4 ) \\ [ \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ] [ \begin{matrix}1 \\ - 1 \\ 0\end{matrix} ] = [ \begin{matrix}{S}_{90 °}^{0} \\ {S}_{90 °}^{1} \\ {S}_{90 °}^{2}\end{matrix} ] & ( 5 ) \\ [ \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ] [ \begin{matrix}1 \\ 0 \\ 1\end{matrix} ] = [ \begin{matrix}{S}_{45 °}^{0} \\ {S}_{45 °}^{1} \\ {S}_{45 °}^{2}\end{matrix} ] & ( 6 ) \\ [ \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ] [ \begin{matrix}1 \\ 0 \\ - 1\end{matrix} ] = [ \begin{matrix}{S}_{135 °}^{0} \\ {S}_{135 °}^{1} \\ {S}_{135 °}^{2}\end{matrix} ] & ( 7 )\end{matrix}$$

Thus, the Mueller matrix M (ωo, ωi) indicated in Expression (8) can be calculated on the basis of Expression (4) to Expression (7). Further, normalization is performed to eliminate influence of luminance in the Mueller matrix M(ωo, ωi). Expression (9) indicates the Mueller matrix M(ωo, ωi) after normalization.

$$\begin{matrix}[ Math . 4 ] & \\ [ \begin{matrix}{m}_{00} = \frac{{S}_{0 °}^{0} + {S}_{90 °}^{0}}{2} & {m}_{01} = \frac{{S}_{0 °}^{0} - {S}_{90 °}^{0}}{2} & {m}_{02} = \frac{{S}_{45 °}^{0} - {S}_{135 °}^{0}}{2} \\ {m}_{10} = \frac{{S}_{0 °}^{1} + {S}_{90 °}^{1}}{2} & {m}_{11} = \frac{{S}_{0 °}^{1} - {S}_{90 °}^{1}}{2} & {m}_{12} = \frac{{S}_{45 °}^{1} - {S}_{135 °}^{1}}{2} \\ {m}_{20} = \frac{{S}_{0 °}^{2} + {S}_{90 °}^{2}}{2} & {m}_{21} = \frac{{S}_{0 °}^{2} - {S}_{90 °}^{2}}{2} & {m}_{22} = \frac{{S}_{45 °}^{2} - {S}_{135 °}^{2}}{2}\end{matrix} ] & ( 8 ) \\ [ \begin{matrix}1 & {m}_{01} / {m}_{00} & {m}_{02} / {m}_{00} \\ {m}_{10} / {m}_{00} & {m}_{11} / {m}_{00} & {m}_{12} / {m}_{00} \\ {m}_{20} / {m}_{00} & {m}_{21} / {m}_{00} & {m}_{22} / {m}_{00}\end{matrix} ] & ( 8 )\end{matrix}$$

The Mueller matrix calculated in this manner indicates polarizing and reflecting characteristics which are peculiar to a material of the measurement object. Further, the polarizing and reflecting characteristics are independent of an external environment, and thus, can be utilized in any location if the characteristics are measured once, and it is not necessary to repeatedly acquire the polarizing and reflecting characteristics.

### 2. Configuration of Material Determination System

The material determination system determines a material of a material determination target on the basis of material polarizing characteristic information indicating polarizing and reflecting characteristics for each incident direction ωi of the incident polarized light and for each emission direction ωo of the reflected light for each material, emitted polarized light information calculated from the polarized image based on the reflected light in the emission direction ωo from the material determination target and incident polarized light information of the incident polarized light in the incident direction ωi.

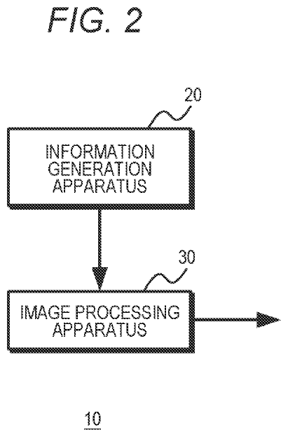

FIG. 2 illustrates an example of a configuration of the material determination system. A material determination system 10 includes an information generation apparatus 20 and an image processing apparatus 30. The information generation apparatus 20 generates material polarizing characteristic information for each material using known materials. Note that the information generation apparatus 20 may register the generated material polarizing characteristic information in a database unit 50 or may output the generated polarizing characteristic information to the image processing apparatus 30. The image processing apparatus 30 captures an image of the material determination target to acquire a polarized image and calculates polarizing and reflecting characteristics on the basis of the polarized image. Further, the image processing apparatus 30 determines the material of the material determination target on the basis of the material polarizing characteristic information acquired from the information generation apparatus 20 or the database unit 50, the calculated polarizing and reflecting characteristics, the incident direction ωi and the emission direction ωo.

### 3. Information Generation Apparatus

### 3-1. Configuration of Information Generation Apparatus

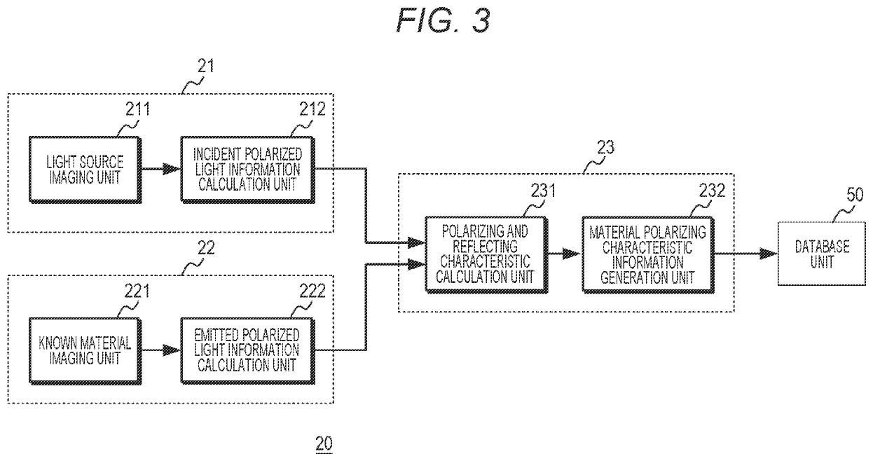

FIG. 3 illustrates an example of a configuration of an information generation apparatus. The information generation apparatus 20 includes a light source information acquisition unit 21, an emitted polarized light information acquisition unit 22, and a material polarizing characteristic information generation unit 23.

The light source information acquisition unit 21 acquires incident polarized light information regarding a light source. The light source information acquisition unit 21 includes a light source imaging unit 211 and an incident polarized light information calculation unit 212.

The light source imaging unit 211 includes an imaging unit and a polarizing plate which can change the polarization direction and which is provided in front of the imaging unit. The light source imaging unit 211 captures images of the light source at the imaging unit for each polarization direction to generate polarized images in a plurality of polarization directions while setting a plurality of predetermined polarization directions as the polarization directions of the polarizing plate (linear polarization) and outputs the polarized images to the incident polarized light information calculation unit 212.

The incident polarized light information calculation unit 212 calculates an incident Stokes vector for each incident direction on the basis of the polarized images generated at the light source imaging unit 211. The incident polarized light information calculation unit 212 outputs the calculated incident Stokes vector for each incident direction, for example, incident Stokes vectors VSi¹ to VSi^{m} in incident directions ωi¹ to ωi^{m} to the material polarizing characteristic information generation unit 23 as the incident polarized light information. Note that the incident direction may be determined by a position of the light source being controlled at the information generation apparatus 20 or position information may be acquired from the light source to determine a direction of the light source with respect to the light source imaging unit 211. Further, the incident polarized light information calculation unit 212 may employ a configuration where information indicating the incident direction is input from outside.

The emitted polarized light information acquisition unit 22 acquires emitted polarized light information regarding the reflected light from the information generation target whose material is obvious in advance. The emitted polarized light information acquisition unit 22 includes a known material imaging unit 221 and an emitted polarized light information calculation unit 222.

The known material imaging unit 221 includes an imaging unit and a polarizing plate which can change the polarization direction and which is provided in front of the imaging unit. The known material imaging unit 221 captures images of the information generation target to generate polarized images for each polarization direction while setting a plurality of predetermined polarization directions as the polarization directions of the polarizing plate (linear polarization). Further, the known material imaging unit 221 performs calibration before imaging and obtains a direction (emission direction ωo) of the reflected light incident on pixels of the known material imaging unit 221 in a camera coordinate system. Note that calibration may be performed using any method, and calibration is performed so that the emission direction ωo can be obtained in the camera coordinate system by using an external parameter and an internal parameter generated through the calibration. Further, the known material imaging unit 221 can capture images of the information generation target from different directions, for example, from directions of different zenith angles and generates polarized images in different emission directions ωo for each of a plurality of polarization directions.

The emitted polarized light information calculation unit 222 calculates the emitted Stokes vector VSo using observation values (pixel values) indicated by the polarized images generated at the known material imaging unit 221. Further, the emitted polarized light information calculation unit 222 calculates the reflected Stokes vector for each emission direction using the polarized images in different polarization directions, which are generated for each emission direction. The emitted polarized light information calculation unit 222 outputs the calculated emitted Stokes vector for each emission direction, for example, emitted Stokes vectors VSo¹ to VSoⁿ in emission directions ωo¹ to ωoⁿ to the material polarizing characteristic information generation unit 23 as the emitted polarized light information.

The material polarizing characteristic information generation unit 23 generates material polarizing characteristic information indicating the polarizing and reflecting characteristics of the information generation target. A polarizing and reflecting characteristic calculation unit 231 and a material polarizing characteristic information generation unit 232 are included.

The polarizing and reflecting characteristic calculation unit 231 calculates the polarizing and reflecting characteristics on the basis of the incident Stokes vectors VSi¹ to VSi^{m} in the incident directions ωi¹ to ωi^{m} indicated in the incident polarized light information supplied from the light source information acquisition unit 21, and the emitted Stokes vectors VSo¹ to VSoⁿ in the emission directions ωo¹ to ωoⁿ indicated in the emitted polarized light information supplied from the emitted polarized light information acquisition unit 22, for example, calculates a Mueller matrix on the basis of Expression (2) for each combination of the incident direction and the emission direction. For example, the polarizing and reflecting characteristic calculation unit 231 calculates a Mueller matrix M(ωo¹, ωi¹) on the basis of the incident Stokes vector VSi¹ in the incident direction ωi¹ and the emitted Stokes vector VSo¹ in the emission direction ωo¹. Further, the polarizing and reflecting characteristic calculation unit 231 calculates a Mueller matrix M(ωoⁿ, ωi^{m}) on the basis of the incident Stokes vector VSi^{m} in the incident direction ωi^{m} and the emitted Stokes vector VSoⁿ in the emission direction ωoⁿ. The material polarizing characteristic information generation unit 23 normalizes the calculated Mueller matrix and outputs the normalized Mueller matrix to the material polarizing characteristic information generation unit 232.

The material polarizing characteristic information generation unit 232 generates material polarizing characteristic information by associating the incident direction and the emission direction with the polarizing and reflecting characteristic information (for example, the normalized Mueller matrix) calculated at the polarizing and reflecting characteristic calculation unit 231. Further, the light source information acquisition unit 21 and the emitted polarized light information acquisition unit 22 acquire the incident polarized light information and the emitted polarized light information for each known material, and the polarizing and reflecting characteristic calculation unit 231 generates polarizing and reflecting characteristic information for each known material and generates material determination information in which the incident direction and the emission direction are associated with the polarizing and reflecting characteristic information for each known material. The material polarizing characteristic information generation unit 232 outputs the generated material polarizing characteristic information to, for example, the database unit 50 as described above. Note that the database unit 50 may be provided at the information generation apparatus 20 or may be provided at the image processing apparatus 30. Further, it may be provided at an external apparatus different from the information generation apparatus 20 and the image processing apparatus 30.

### 3-2. Operation of Information Generation Apparatus

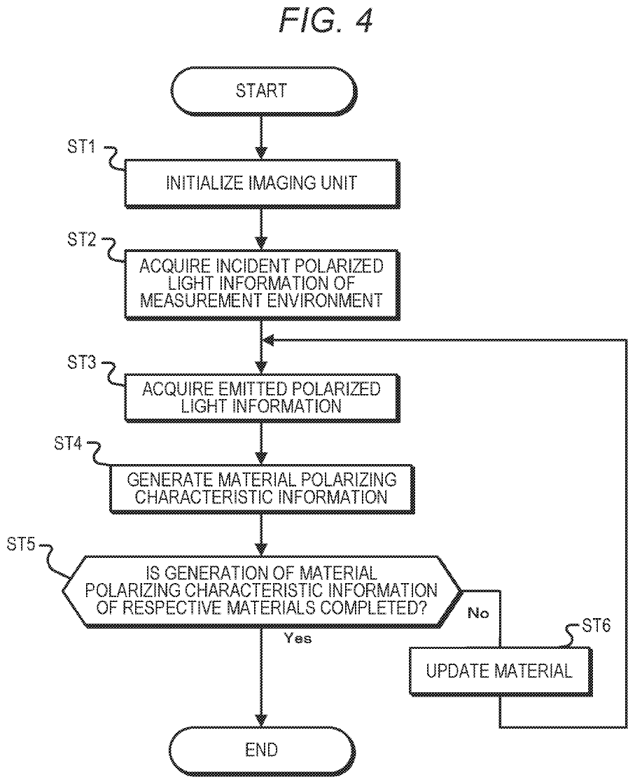

Subsequently, operation of the information generation apparatus 20 will be described. FIG. 4 is a flowchart illustrating an example of operation of the information generation apparatus. In step ST1, the information generation apparatus 20 initializes the imaging unit. The information generation apparatus 20 initializes the light source imaging unit 211 of the light source information acquisition unit 21 and the known material imaging unit 221 of the emitted polarized light information acquisition unit 22. The information generation apparatus 20 calibrates the light source imaging unit 211 and the known material imaging unit 221 so that coordinate systems match each other, and the processing proceeds to step ST2.

In step ST2, the information generation apparatus 20 acquires incident polarized light information of the measurement environment. The light source information acquisition unit 21 of the information generation apparatus 20 acquires incident directions and incident polarized light information indicating an incident Stokes vector for each incident direction, and the processing proceeds to step ST3.

In step ST3, the information generation apparatus 20 acquires emitted polarized light information. The emitted polarized light information acquisition unit 22 of the information generation apparatus 20 acquires emitted polarized light information indicating an emitted Stokes vector for each emitted direction, for each incident direction of the light source, and the processing proceeds to step ST4.

In step ST4, the information generation apparatus 20 generates material polarizing characteristic information. The material polarizing characteristic information generation unit 23 of the information generation apparatus 20 generates polarizing and reflecting characteristics for each combination of the incident direction and the emission direction on the basis of the incident polarized light information acquired in step ST2 and the emitted polarized light information acquired in step ST3. For example, the material polarizing characteristic information generation unit 23 calculates a Mueller matrix M(ω, ωi) for each incident direction and for each emission direction using the incident Stokes vector VSi in the incident direction ωi and the emitted Stokes vector VSo in the emission direction ω. Further, the material polarizing characteristic information generation unit 23 generates material polarizing characteristic information by associating the incident direction and the emission direction with the polarizing and reflecting characteristic information, and the processing proceeds to step ST5.

In step ST5, the information generation apparatus 20 determines whether generation of the material polarizing characteristic information of respective materials has been completed. In a case where there is a material for which material polarizing characteristic information has not been generated, the processing proceeds to step ST6, and in a case where generation of the material polarizing characteristic information of respective materials has been completed, the information generation apparatus 20 finishes the processing.

In step ST6, the information generation apparatus 20 updates a material. The information generation apparatus 20 polarizes a subject for which an image is to be captured at the known material imaging unit 221 of the emitted polarized light information acquisition unit 22 to a material for which material polarizing characteristic information has not been generated, and the processing returns to step ST3.

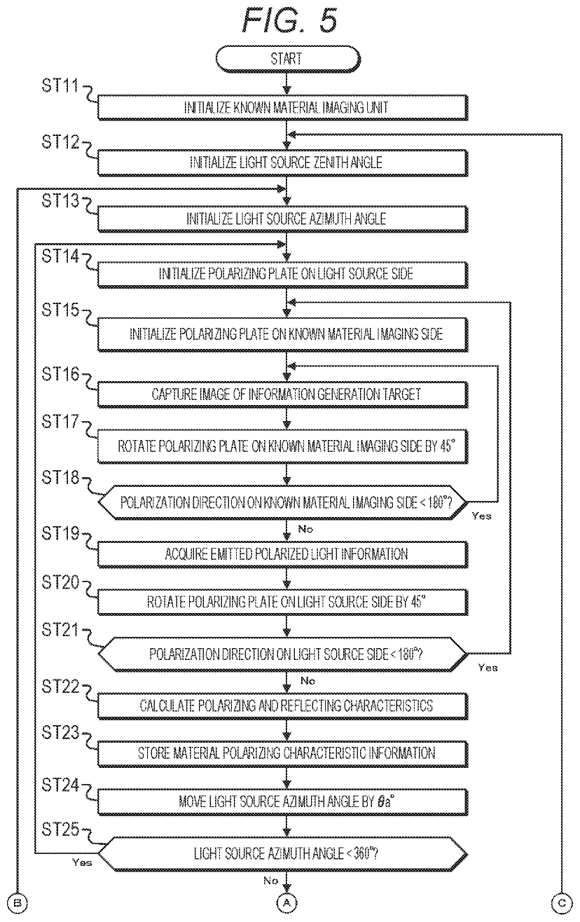

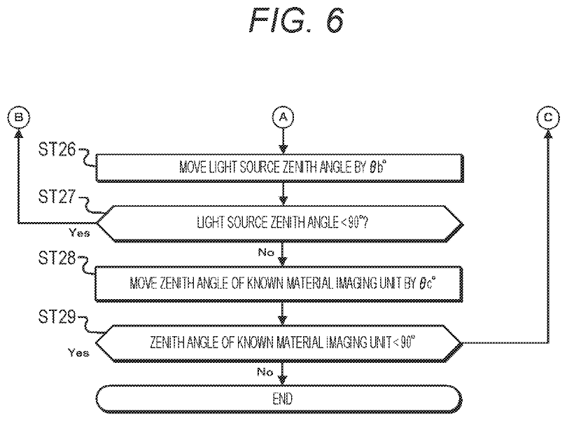

Further, FIG. 5 and FIG. 6 are flowcharts illustrating the operation of the information generation apparatus in detail. FIG. 5 and FIG. 6 indicate a case where an incident direction and an incident Stokes vector at a position for each angle θa in an azimuth direction and for each angle θb in a zenith direction, are used. Further, an imaging direction of the known material imaging unit 221 is moved for each angle θc in the zenith direction. Still further, the light source imaging unit 211 and the known material imaging unit 221 switch the polarization direction to “0°, 45°, 90° and 135°”. Note that the incident polarized light information is acquired in advance.

In step ST11, the information generation apparatus 20 initializes the known material imaging unit. The emitted polarized light information acquisition unit 22 of the information generation apparatus 20 calibrates the known material imaging unit 221 to set an azimuth angle and a zenith angle at 0°, and the processing proceeds to step ST12.

In step ST12, the information generation apparatus 20 initializes the light source zenith angle. The light source information acquisition unit 21 of the information generation apparatus 20 initializes the light source imaging unit 211 and sets a direction in which the zenith angle of the known material imaging unit 221 is 0° as a direction in which the zenith angle of the light source imaging unit 211 is 0°, and the processing proceeds to step ST13.

In step ST13, the information generation apparatus 20 initializes a light source azimuth angle. The light source information acquisition unit 21 of the information generation apparatus 20 initializes the light source imaging unit 211 and sets a direction in which the direction of the known material imaging unit 221 is 0° as a direction in which the azimuth angle of the light source imaging unit 211 is 0°, and the processing proceeds to step ST14.

In step ST14, the information generation apparatus 20 initializes the polarizing plate on the light source side. The light source information acquisition unit 21 of the information generation apparatus 20 sets the polarization direction of the polarizing plate used at the light source imaging unit 211 at 0°, and the processing proceeds to step ST15.

In step ST15, the information generation apparatus 20 initializes the polarizing plate on the known material imaging side. The emitted polarized light information acquisition unit 22 of the information generation apparatus 20 sets the polarization direction of the polarizing plate used at the known material imaging unit 221 at 0°, and the processing proceeds to step ST16.

In step ST16, the information generation apparatus 20 captures an image of the information generation target. The known material imaging unit 221 captures an image of the information generation target whose material is obvious to generate a polarized image, and the processing proceeds to step ST17.

In step ST17, the information generation apparatus 20 rotates the polarizing plate on the known material imaging side by 45°. The emitted polarized light information acquisition unit 22 of the information generation apparatus 20 rotates the polarization direction of the polarizing plate by 45°, and the processing proceeds to step ST18.

In step ST18, the information generation apparatus 20 determines whether the polarization direction on the known material imaging side is smaller than 180°. The processing of the emitted polarized light information acquisition unit 22 of the information generation apparatus 20 returns to step ST16 in a case where the polarization direction after rotation is smaller than 180°, and proceeds to step ST19 in a case where the polarization direction after rotation is equal to or greater than 180°.

In step ST19, the information generation apparatus 20 acquires emitted polarized light information. Respective polarized images in a case where the polarization direction is “0°, 45°, 90° and 135°” are generated by the processing from step ST16 to step ST18 being performed, and thus, the information generation apparatus 20 calculates emitted Stokes vectors on the basis of the generated polarized images, and the processing proceeds to step ST20.

In step ST20, the information generation apparatus 20 rotates the polarizing plate on the light source side by 45°. The light source information acquisition unit 21 of the information generation apparatus 20 rotates the polarization direction of the polarizing plate by 45°, and the processing proceeds to step ST21.

In step ST21, the information generation apparatus 20 determines whether the polarization direction on the light source side is smaller than 180°. The processing of the light source information acquisition unit 21 of the information generation apparatus 20 returns to step ST15 in a case where the polarization direction after rotation is smaller than 180°, and proceeds to step ST22 in a case where the polarization direction after rotation is equal to or greater than 180°.

In step ST22, the information generation apparatus 20 calculates polarizing and reflecting characteristics. The light source information acquisition unit 21 of the information generation apparatus 20 calculates a Mueller matrix on the basis of the emitted Stokes vectors in a case where the polarization direction of the incident polarized light on the information generation target is “0°, 45°, 90° and 135°”. In other words, the light source information acquisition unit 21 calculates a Mueller matrix indicated in Expression (8) on the basis of the above-described Expressions (4) to (7), and the processing proceeds to step ST23.

In step ST23, the information generation apparatus 20 stores the material polarizing characteristic information. The light source information acquisition unit 21 of the information generation apparatus 20 generates the material polarizing characteristic information in which the incident direction ωi indicating a direction of the light source and the emission direction ωo indicating a direction of the known material imaging unit 221 are associated with the Mueller matrix calculated in step ST22 and stores the material polarizing characteristic information in a database unit, or the like, and the processing proceeds to step ST24.

In step ST24, the information generation apparatus 20 moves the light source azimuth angle by θa°. The light source information acquisition unit 21 of the information generation apparatus 20 moves an azimuth angle of the light source imaging unit 211 by θa°, and the processing proceeds to step ST25.

In step ST25, the information generation apparatus 20 determines whether the light source azimuth angle is smaller than 360°. The processing of the light source information acquisition unit 21 of the information generation apparatus 20 returns to step ST14 in a case where the light source azimuth angle is smaller than 360°, and proceeds to step ST26 in a case where the light source azimuth angle is equal to or greater than 360°.

In step ST26, the information generation apparatus 20 moves the light source zenith angle by θb°. The light source information acquisition unit 21 of the information generation apparatus 20 moves a zenith angle of the light source imaging unit 211 by θb°, and the processing proceeds to step ST27.

In step ST27, the information generation apparatus 20 determines whether a light source zenith angle is smaller than 90°. The processing of the light source information acquisition unit 21 of the information generation apparatus 20 returns to step ST13 in a case where the light source zenith angle is smaller than 90°, and proceeds to step ST28 in a case where the light source zenith angle is equal to or greater than 90°. In other words, the material polarizing characteristic information for each incident direction in which resolution in the azimuth direction is θa° and resolution in the zenith direction is θb° is stored in a database unit, or the like, for one emission direction by the processing from step ST13 to step ST27 being performed.

In step ST28, the information generation apparatus 20 moves the zenith angle of the known material imaging unit by θc°. The emitted polarized light information acquisition unit 22 of the information generation apparatus 20 moves the zenith angle of the known material imaging unit 221 by θc°, and the processing proceeds to step ST29.

In step ST29, the information generation apparatus 20 determines whether the zenith angle of the known material imaging unit is smaller than 90°. The processing of the emitted polarized light information acquisition unit 22 of the information generation apparatus 20 returns to step ST12 in a case where the zenith angle of the known material imaging unit 221 is smaller than 90°, and the emitted polarized light information acquisition unit 22 finishes the processing in a case where the zenith angle is equal to or greater than 90°. Thus, the material polarizing characteristic information for each incident direction in which resolution in the azimuth direction is an angle θa and resolution in the zenith direction is an angle θb and for each emission direction in which resolution in the zenith direction is an angle θc is stored in a database unit, or the like.

Further, by the processing illustrated in FIG. 5 and FIG. 6 being performed for each of information generation targets whose materials are different, the material polarizing characteristic information for each incident direction in which resolution in the azimuth direction is an angle θa and resolution in the zenith direction is an angle θb and for each emission direction in which resolution in the zenith direction is an angle θc can be stored in a database unit, or the like, for each material.

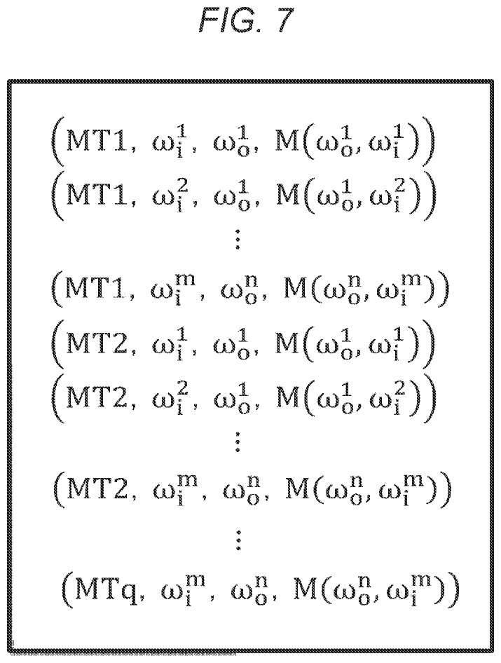

In this manner, according to the information generation apparatus of the present technology, it is possible to generate material polarizing characteristic information indicating polarizing and reflecting characteristics which are independent of an external environment and which are peculiar to a material. FIG. 7 illustrates an example of the material polarizing characteristic information and illustrates, for example, Mueller matrixes M(ωo¹, ωi¹) to M(ωoⁿ, ωi^{m}) for each combination of incident directions ωi¹ to ωi^{m} and emission directions ωo¹ to ωoⁿ for a material MT1. Further, FIG. 7 illustrates Mueller matrixes M(ωo¹, ωi¹) to M(ωoⁿ, ωi^{m}) for each combination of incident directions ωi¹ to ωi^{m} and emission directions ωo¹ to ωoⁿ for a material MT2. Note that FIG. 7 illustrates material polarizing characteristics of q types of different materials for each combination of the incident direction and the emission direction.

### 4. Image Processing Apparatus

### 4-1. Configuration of Image Processing Apparatus

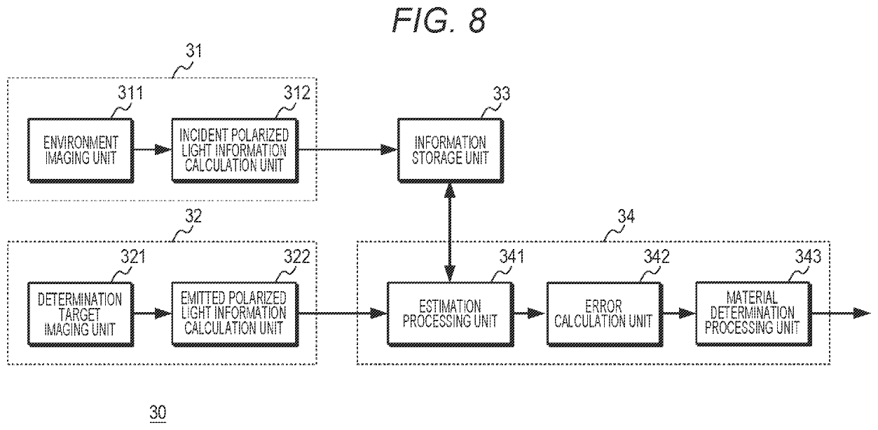

A configuration of the image processing apparatus will be described next. FIG. 8 illustrates an example of a configuration of an image processing apparatus. The image processing apparatus 30 includes a determination environment information acquisition unit 31, a determination target information acquisition unit 32, an information storage unit 33 and a determination processing unit 34.

The determination environment information acquisition unit 31 acquires incident polarized light information of a light source in a material determination environment. The determination environment information acquisition unit 31 includes an environment imaging unit 311 and an incident polarized light information calculation unit 312.

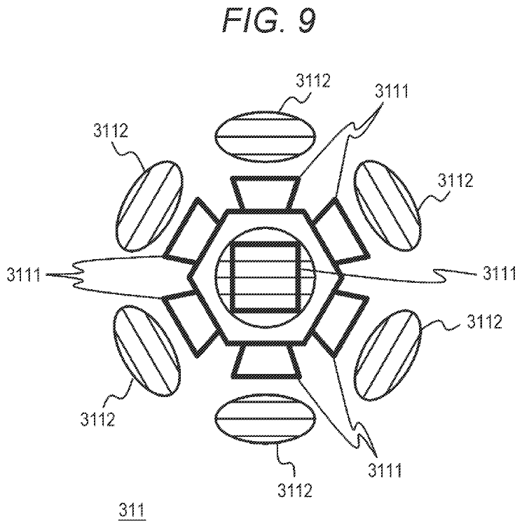

FIG. 9 illustrates an example of a configuration of an environment imaging unit 311. The environment imaging unit 311 includes, for example, a plurality of imaging units 3111 having different imaging directions and polarizing plates 3112 which can change the polarization direction, and which are provided in front of the respective imaging units. Note that the respective polarizing plates 3112 have the same polarization directions. The environment imaging unit 311 captures an image of an environment when an image of the material determination target is captured to generate, for example, a full spherical polarized image for each of a plurality of polarization directions. Note that the environment imaging unit 311 may acquire a full spherical polarized image for each of a plurality of polarization directions with one imaging unit 3111 and one polarizing plate 3112 using a fish-eye lens, or the like. Further, the environment imaging unit 311 does not always generate a full spherical polarized image. For example, in a case where light sources are provided only in a limited range, the environment imaging unit 311 may generate a polarized image in the limited range.

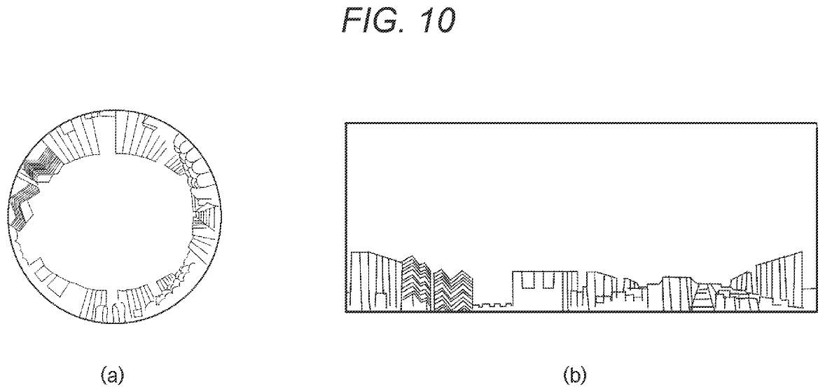

FIG. 10 illustrates an example of a polarized image generated at the environment imaging unit 311. Note that (a) of FIG. 10 illustrates an example of a fisheye image indicating a full sphere, and (b) of FIG. 10 illustrates an example of a developed image obtained by developing the fisheye image on a cylindrical surface.

The incident polarized light information calculation unit 312 divides the polarized image generated at the environment imaging unit 311 in the zenith direction and in the azimuth direction, calculates an average incident Stokes vector within a region and an average incident direction of light beam for each of divided regions to set the average incident Stokes vector and the average incident direction as incident polarized light information, and outputs the incident polarized light information to the information storage unit 33.

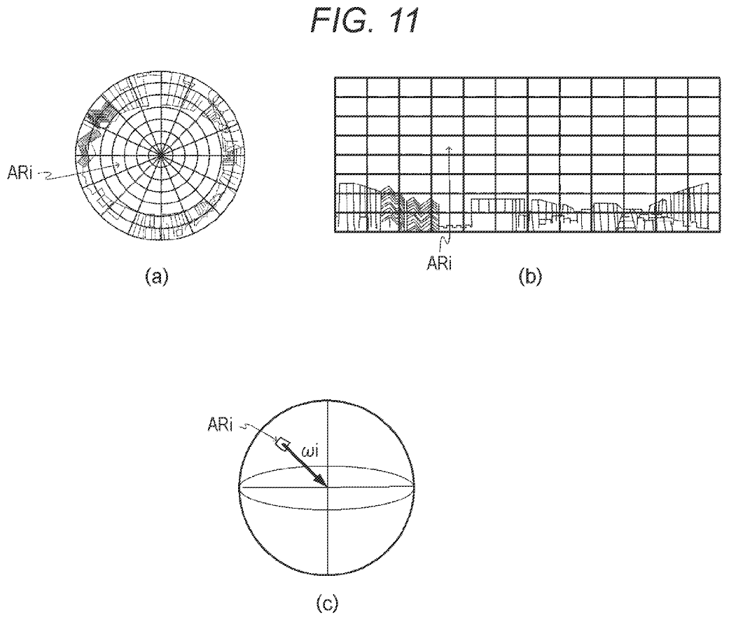

FIG. 11 illustrates an example of division of the polarized image. Note that (a) of FIG. 11 illustrates an example of division of the fisheye image illustrated in (a) of FIG. 10, and (b) of FIG. 11 illustrates an example of division of the developed image illustrated in (b) of FIG. 10.

Here, as illustrated in (c) of FIG. 11, in a case where a light source position of the incident polarized light in the incident direction ωi is included in a region ARi, the incident polarized light information calculation unit 312 calculates an average incident direction ωi¹ and an average incident Stokes vector VSi¹ in the region ARi. The incident polarized light information calculation unit 312 calculates average incident directions and average incident Stokes vectors for other regions in a similar manner. The incident polarized light information calculation unit 312 outputs the calculated incident Stokes vector for each incident direction to the information storage unit 33.

Further, the determination environment information acquisition unit 31 calculates an incident Stokes vector for each light source when an image of the material determination target is captured, and outputs incident polarized light information which indicates the incident Stokes vector of each incident direction for each light source to the information storage unit 33.

The determination target information acquisition unit 32 acquires emitted polarized light information regarding the reflected light from the material determination target. The determination target information acquisition unit 32 includes a determination target imaging unit 321 and an emitted polarized light information calculation unit 322.

The determination target imaging unit 321 includes an imaging unit and a polarizing plate which can change the polarization direction and which are provided in front of the imaging unit. The determination target imaging unit 321 captures an image of the material determination target from the emission direction ωo to generate a polarized image for each of a plurality of polarization directions. Further, the determination target imaging unit 321 performs calibration before imaging in a similar manner to the above-described known material imaging unit 221, or the like, so that the emission direction ωo can be obtained in the camera coordinate system by using an external parameter and an internal parameter generated through the calibration.

The emitted polarized light information calculation unit 322 calculates the emitted Stokes vector VSo using observation values (pixel values) indicated by the polarized images generated at the determination target imaging unit 321. Further, the emitted polarized light information calculation unit 222 outputs the emitted polarized light information indicating the emission direction ωo and the emitted Stokes vector VSo to the determination processing unit 34.

The information storage unit 33 stores the material polarizing characteristic information generated at the information generation apparatus 20 and the incident polarized light information acquired at the determination environment information acquisition unit 31. Note that the information storage unit 33 may store the material polarizing characteristic information acquired from the information generation apparatus 20 or may store the material polarizing characteristic information acquired from a database unit.

The determination processing unit 34 determines the material of the material determination target on the basis of the incident polarized light information acquired at the determination environment information acquisition unit 31, the emitted polarized light information acquired at the determination target information acquisition unit 32, and the material polarizing characteristic information which is generated in advance and which indicates the polarizing and reflecting characteristics for each incident direction of the incident polarized light and for each emission direction of the reflected polarized light. The determination processing unit 34 includes an estimation processing unit 341, an error calculation unit 342, and a material determination processing unit 343.

The estimation processing unit 341 estimates one of the incident polarized light information and the emitted polarized light information using the material polarizing characteristic information selected in accordance with the incident direction of the incident polarized light on the material determination target and the emission direction of the reflected light from the material determination target and the other of the incident polarized light information acquired at the determination environment information acquisition unit and the emitted polarized light information acquired at the determination target information acquisition unit.

The estimation processing unit 341 acquires the incident polarized light information acquired at the determination environment information acquisition unit 31 from the information storage unit 33. Further, the estimation processing unit 341 acquires the material polarizing characteristic information corresponding to the incident direction ωi of the incident polarized light information acquired at the determination environment information acquisition unit 31 and the emission direction ωo of the reflected light incident on the determination target imaging unit 321 from the information storage unit 33. The estimation processing unit 341 estimates the emitted polarized light information on the basis of the acquired incident polarized light information and material polarizing characteristic information or estimates the incident polarized light information on the basis of the acquired material polarizing characteristic information and the emitted polarized light information acquired at the determination target information acquisition unit 32.

For example, the estimation processing unit 341 acquires a Mueller matrix M(ωo, ωi) for each material corresponding to the incident direction ωi and the emission direction ωo of the emitted polarized light information acquired at the determination target information acquisition unit 32 from the information storage unit 33. Further, the estimation processing unit 341 acquires the incident Stokes vector VSi(ωi) in the incident direction ωi from the information storage unit 33. The estimation processing unit 341 calculates the estimated emitted Stokes vector VSEo(ωo) for which the incident direction is not limited to a specific direction on the basis of the Mueller matrix M(ωo, ωi) and the incident Stokes vector VSi(ωi) or calculates the estimated incident Stokes vector VSEi(ωi) on the basis of an inverse matrix of the Mueller matrix M(ωo, ωi) and the emitted Stokes vector VSo(ωo), for each material, and outputs the estimated emitted Stokes vector VSEo(ωo) and the estimated incident Stokes vector VSEi(ωi) to the error calculation unit 342.

The error calculation unit 342 calculates an error between the emitted polarized light information estimated at the estimation processing unit 341 and the emitted polarized light information acquired at the determination target information acquisition unit 32 or an error between the incident polarized light information estimated at the estimation processing unit 341 and the incident polarized light information in the incident direction ωi acquired at the determination environment information acquisition unit 31, for each material. For example, the error calculation unit 342 calculates an integrated value of an error between the estimated emitted Stokes vector VSEo(ωo) estimated at the estimation processing unit 341 and the emitted Stokes vector VSo(ωo) acquired at the determination target information acquisition unit 32 or an error between the estimated incident Stokes vector VSEi(ωi) estimated at the estimation processing unit 341 and the incident Stokes vector VSi(ωi) for each incident direction, for each material and outputs the integrated value to the material determination processing unit 343.

The material determination processing unit 343 determines the material of the material determination target on the basis of the error (or the integrated value of the error) calculated at the error calculation unit 342. For example, the material determination processing unit 343 determines a material for which the error calculated for each material at the error calculation unit 342 becomes a minimum error, as the material of the material determination target. Further, the determination processing unit may determine a material with a minimum error as the material of the material determination target in a case where the minimum error in the calculated error is smaller than a threshold set in advance. Further, the determination processing unit may calculate an error for a specific material and may determine the specific material as the material of the material determination target in a case where the calculated error is smaller than a threshold set in advance. In other words, the determination processing unit can also determine whether or not the material is a desired material as well as determine the material.

### 4-2. Operation of Image Processing Apparatus

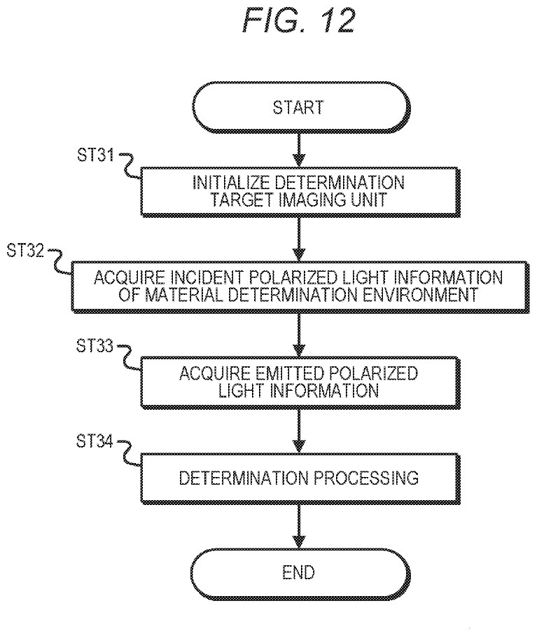

FIG. 12 is a flowchart illustrating an example of operation of the image processing apparatus. In step ST31, the image processing apparatus 30 initializes the determination target imaging unit. The image processing apparatus 30 initializes the determination target imaging unit 321 of the determination target information acquisition unit 32. The image processing apparatus 30 calibrates the determination target imaging unit 321 so that coordinate systems match, and the processing proceeds to step ST32.

In step ST32, the image processing apparatus 30 acquires incident polarized light information of a material determination environment. The determination environment information acquisition unit 31 of the image processing apparatus 30 acquires incident polarized light information of the light source on the basis of the polarized image obtained by capturing an image of the material determination environment, and the processing proceeds to step ST33.

In step ST33, the image processing apparatus 30 acquires emitted polarized light information. The determination target information acquisition unit 32 of the image processing apparatus 30 acquires the emitted polarized light information indicating the emitted Stokes vector of the material determination target and the emission direction, and the processing proceeds to step ST34.

In step ST34, the image processing apparatus 30 performs determination processing. The determination processing unit 34 of the image processing apparatus 30 determines the material of the material determination target on the basis of the incident direction detected in step ST32, the emitted polarized light information acquired in step ST33, and the material polarizing characteristic information and the incident polarized light information stored in advance in the information storage unit 33.

### 4-2-1. First Operation of Determination Processing Unit

In first operation of the determination processing unit 34, the emitted polarized light information is estimated on the basis of the polarizing and reflecting characteristic information corresponding to a plurality of incident directions ωi when an image of the material determination target is captured and the emission direction ωo of the reflected light supplied to the determination target imaging unit 321, and the incident polarized light information in the incident direction ωi acquired at the determination environment information acquisition unit 31, and the material of the material determination target is determined on the basis of the estimated emitted polarized light information and the emitted polarized light information acquired at the determination target information acquisition unit 32.

The determination processing unit 34 performs operation of Expression (10) using the incident Stokes vector VSi(ωi) in the incident direction ωi and the Mueller matrix M(ωo, ωi) acquired from the information storage unit 33 and calculates the estimated emitted Stokes vector VSeo(ωo) in the emission direction ωo.

$$M ( ω o , ω i ) \cdot VSi ( ω i ) = VSeo ( ω o ) \qquad ( 10 )$$

Further, the determination processing unit 34 calculates the estimated emitted Stokes vector VSEo(ωo) for which the incident direction is not limited to a specific direction by performing operation indicated in Expression (11) and integrating the estimated emitted Stokes vectors VSeo(ωo) for each of a plurality of incident directions ωi.

$$\begin{matrix}[ Math . 5 ] & \\ {\int }_{{ω}_{i}} M ( {ω}_{o} , {ω}_{i} ) \cdot VSi ( {ω}_{i} ) = {\int }_{{ω}_{i}} VSeo ( {ω}_{o} ) = VSEo ( {ω}_{o} ) & ( 11 )\end{matrix}$$

The determination processing unit 34 calculates an error E(ωo) between the estimated emitted Stokes vector VSEo(ωo) and the emitted Stokes vector VSo(ωo) acquired at the determination target information acquisition unit 32 on the basis of Expression (12). Further, a normalized Stokes vector may be used in calculation of the error E(ωo) so as to prevent influence of luminance components. Note that Expression (13) indicates a Stokes vector before normalization, and Expression (14) indicates a Stokes vector after normalization.

$$\begin{matrix}[ Math . 6 ] & \\ E ( {ω}_{o} ) = ‖ VSo ( {ω}_{o} ) - VSEo ( {ω}_{o} ) ‖ & ( 12 ) \\ [ \begin{matrix}{S}^{0} \\ {S}^{1} \\ {S}^{2}\end{matrix} ] & ( 13 ) \\ [ \begin{matrix}1 \\ {S}^{1} / {S}^{0} \\ {S}^{2} / {S}^{0}\end{matrix} ] & ( 14 )\end{matrix}$$

The determination processing unit 34 performs the above-described processing using the Mueller matrix for each material, compares errors E(ωo)−1 to E(ωo)−q (where q is the number of materials) calculated for each material and determines the smallest error E(ωo)min. Further, in a case where the error E(ωo)min is smaller than a threshold Tho set in advance, the determination processing unit 34 determines a material corresponding to the Mueller matrix used to calculate the error E(ωo)min as the material of the material determination target. Further, in a case where the error E(ωo)min is equal to or greater than the threshold Tho, the determination processing unit 34 determines that the material of the material determination target cannot be determined. Note that determination accuracy of the material can be adjusted by adjusting the threshold Tho. Through the processing as described above, it is possible to determine the material of the material determination target.

### 4-2-2. Second Operation of Determination Processing Unit

In second operation of the determination processing unit 34, the incident polarized light information is estimated on the basis of the polarizing and reflecting characteristic information corresponding to a plurality of incident directions ωi when an image of the material determination target is captured and the emission direction ωo of the reflected light supplied to the determination target imaging unit 321, and the emitted polarized light information acquired at the determination target information acquisition unit 32, and the material of the material determination target is determined on the basis of the estimated incident polarized light information and the incident polarized light information for each incident direction acquired at the determination environment information acquisition unit 31.

The determination processing unit 34 performs operation of Expression (15) using an inverse matrix M⁻¹ (ωo, ωi) of the Mueller matrix corresponding to the incident direction ωi and the emission direction ωo and the emitted Stokes vector VSo(ωo) acquired at the determination target information acquisition unit 32 and calculates the estimated incident Stokes vector VSEi(ωi) in the incident direction ωi.

$$V S E i ( ω i ) = {M}^{- 1} ( ω o , ω i ) \cdot VSo ( ω o ) \qquad ( 15 )$$

The determination processing unit 34 calculates an error e(ωo, ωi) between the incident Stokes vector VSi(ωi) in the incident direction ωi and the estimated incident Stokes vector VSEi(ωi) on the basis of Expression (16). Further, a normalized Stokes vector is used in calculation of the error e(ωo, ωi) so as to prevent influence of luminance components. Still further, the determination processing unit 34 calculates an error E(ωi) in a case where the incident direction is not limited to a specific direction by performing operation indicated in Expression (17) and integrating the errors e(ωo, ωi) calculated for each of a plurality of incident directions ωi.

$$\begin{matrix}[ Math . 7 ] & \\ e ( {ω}_{o} , {ω}_{i} ) = ‖ VSi ( {ω}_{i} ) - V S E i ( {ω}_{i} ) ‖ & ( 16 ) \\ E ( {ω}_{o} ) = {\int }_{{ω}_{i}} e ( {ω}_{o} , {ω}_{i} ) = {\int }_{{ω}_{i}} ‖ VSi ( {ω}_{i} ) - V S E i ( {ω}_{i} ) ‖ = {\int }_{{ω}_{i}} ‖ VSi ( {ω}_{i} ) - {M}^{- 1} ( {ω}_{o} , {ω}_{i} ) \cdot VSo ( {ω}_{o} ) ‖ & ( 17 )\end{matrix}$$

The determination processing unit 34 performs the above-described processing using the Mueller matrix for each material, compares errors E(ωi)−1 to E(ωi)−q (where q is the number of materials) calculated for each material and determines the smallest error E(ωi)min. Further, in a case where the error E(ωi)min is smaller than a threshold Thi set in advance, the determination processing unit 34 determines a known material corresponding to the Mueller matrix used to calculate the error E(ωi)min as the material of the material determination target. Further, in a case where the error E(ωi)min is equal to or greater than the threshold Thi, the determination processing unit 34 determines that the material of the material determination target cannot be determined. Note that determination accuracy of the material can be changed by adjusting the threshold Thi. Through the processing as described above, it is possible to determine the material of the material determination target.

In this manner, according to the image processing apparatus of the present technology, it is possible to determine the material of the material determination target on the basis of the material polarizing characteristic information which is generated at the information generation apparatus and which indicates the polarizing and reflecting characteristics peculiar to the material, the incident polarized light information of the light source in the material determination environment and the emitted polarized light information acquired from the polarized image of the material determination target.

### 5. Operation Example of Image Processing Apparatus

### 5-1. First Operation Example

In a first operation example, Mueller matrixes of a plurality of (q types of) materials and incident Stokes vectors of a plurality of (r types of) light sources are stored in the information storage unit 33.

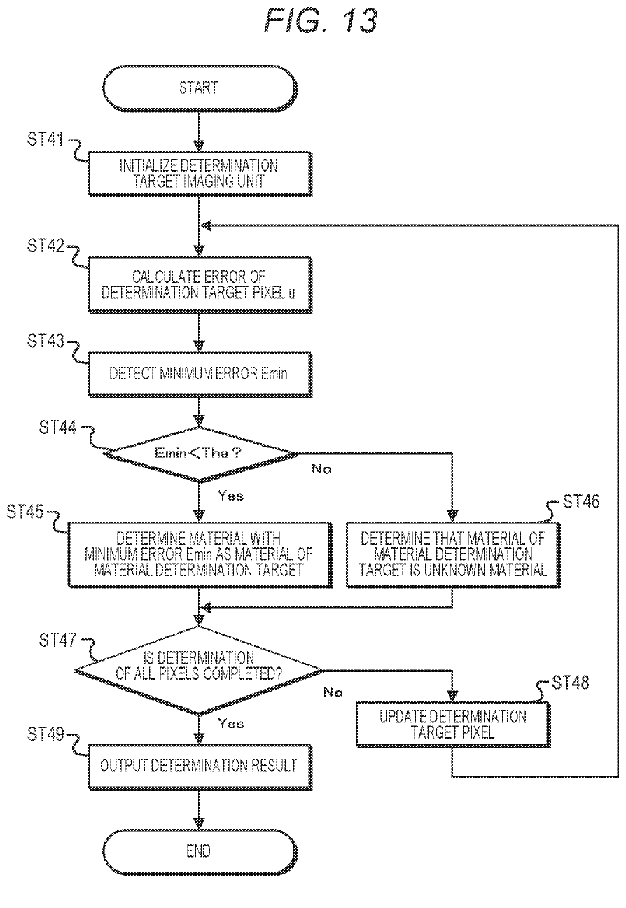

FIG. 13 is a flowchart illustrating a first operation example. In step ST41, the image processing apparatus initializes the determination target imaging unit. The determination target information acquisition unit 32 of the image processing apparatus 30 calibrates the determination target imaging unit 321, and the processing proceeds to step ST42.

In step ST42, the image processing apparatus calculates an error of a determination target pixel u. The determination processing unit 34 of the image processing apparatus 30 calculates an error eij for each combination of an i-th (where i=0, 1, . . . q) type of a material and a j-th (where j=0, 1, . . . , r) type of a light source, and the processing proceeds to step ST43.

In step ST43, the image processing apparatus detects a minimum error Emin. The determination processing unit 34 of the image processing apparatus 30 calculates a combination x, y of a material and a light source for which the error becomes a minimum on the basis of Expression (18) and sets the error of the combination x, y of the material and the light source as a minimum error Emin as indicated in Expression (19).

$$\begin{matrix}[ Math . 8 ] & \\ x , y = {argmin}_{i , j} \{ {e}_{i j} ( {ω}_{o} ) \vert i = 0 , 1 , … , q , j = 0 , 1 , … , r \} & ( 18 ) \\ E min = {e}_{x , y} & ( 19 )\end{matrix}$$

In step ST44, the image processing apparatus determines whether the minimum error Emin is smaller than a threshold Tha. The processing of the determination processing unit 34 of the image processing apparatus 30 proceeds to step ST45 in a case where it is determined that the minimum error Emin detected in step ST43 is smaller than the threshold Tha, and proceeds to step ST46 in a case where the minimum error Emin is equal to or greater than the threshold Tha.

In step ST45, the image processing apparatus determines a material with the minimum error Emin as the material of the material determination target, and the processing proceeds to step ST47.

In step ST46, the image processing apparatus determines that the material of the material determination target is an unknown material, and the processing proceeds to step ST47.

In step ST47, the image processing apparatus determines whether determination of all pixels has been completed. The processing of the image processing apparatus 30 proceeds to step ST48 in a case where determination of all pixels has not been finished, and proceeds to step ST49 in a case where determination of all pixels has been completed.

In step ST48, the image processing apparatus updates the determination target pixel. The image processing apparatus 30 selects a new pixel for which determination of the material has not been performed as the determination target pixel, and the processing returns to step ST42.

In step ST49, the image processing apparatus outputs a determination result. The image processing apparatus 30, for example, displays image regions for which materials are determined as the same material with the same color, the same luminance, or the like, on the basis of the determination result of the material. Further, the image processing apparatus 30 displays regions for which materials are determined as different materials with different color or luminance.

In this manner, according to the first operation example, it is possible to determine materials of respective subjects included in an imaging range of the determination target imaging unit 321. Further, a difference in material is indicated as a difference in attribute of display, so that it is possible to easily determine the difference in material.

### 5-2. Second Operation Example

In a second operation example, Mueller matrixes of a plurality of (q types of) materials and incident Stokes vectors of a plurality of (r types of) light sources are stored in the information storage unit 33. Further, unlike with the first operation example, a light source is selected in accordance with a condition when the material is determined, and the material is determined using incident polarized light information of the selected light source.

For example, in a case where it is determined as indoors, incident polarized light information of an indoor illumination light source is used. Further, in a case where the material is determined outdoors, incident polarized light information in a case where the light source is the sun is used. Still further, in a case where the material is determined outdoors at night, incident polarized light information of a light source which illuminates a position of a polarized light determination target is used.

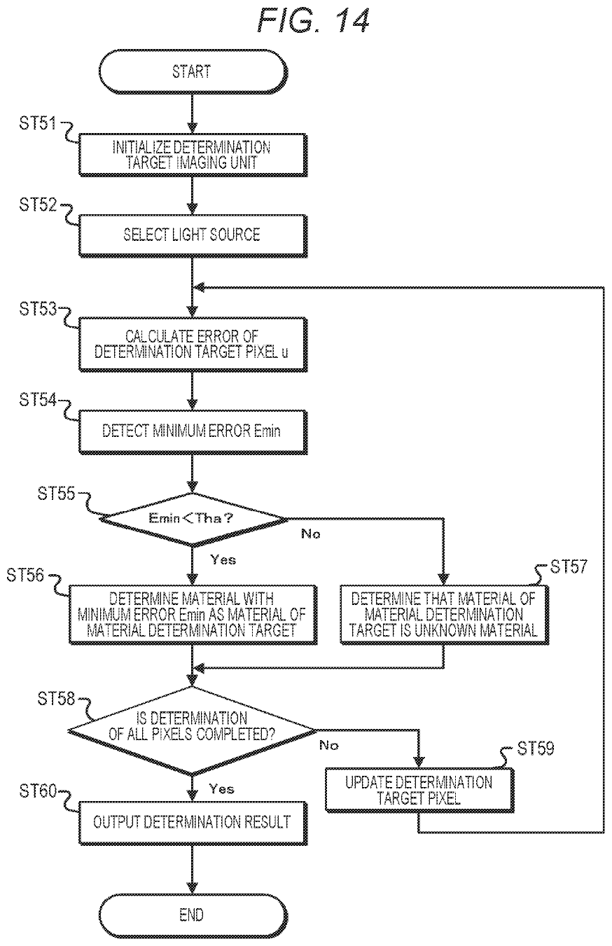

FIG. 14 is a flowchart illustrating the second operation example. In step ST51, the image processing apparatus initializes the determination target imaging unit. The determination target information acquisition unit 32 of the image processing apparatus 30 calibrates the determination target imaging unit 321, and the processing proceeds to step ST52.

In step ST52, the material determination unit selects a light source. The determination environment information acquisition unit 31 of the image processing apparatus 30 selects a light source in accordance with a condition when an image of the material determination target is captured, and the processing proceeds to step ST53.

In step ST53, the image processing apparatus calculates an error of a determination target pixel u. The determination processing unit 34 of the image processing apparatus 30 calculates an error ei for each material of an i-th (where i=0, 1, . . . q) type, and the processing proceeds to step ST54.

In step ST54, the image processing apparatus detects a minimum error Emin. The determination processing unit 34 of the image processing apparatus 30 detects a material x for which an error becomes a minimum on the basis of Expression (20) and sets the error of the material x as a minimum error Emin as indicated in Expression (21). Note that “y” indicates the selected light source.

$$\begin{matrix}[ Math . 9 ] & \\ x = {arg min}_{i} \{ {e}_{i y} ( {ω}_{o} ) \vert i = 0 , 1 , … , q \} & ( 20 ) \\ E min = {e}_{x , y} & ( 21 )\end{matrix}$$

In step ST55, the image processing apparatus determines whether the minimum error Emin is smaller than a threshold Tha. The processing of the determination processing unit 34 of the image processing apparatus 30 proceeds to step ST56 in a case where it is determined that the minimum error Emin detected in step ST54 is smaller than the threshold Tha, and proceeds to step ST57 in a case where the minimum error Emin is equal to or greater than the threshold Tha.

In step ST56, the image processing apparatus determines a material with the minimum error Emin as the material of the material determination target, and the processing proceeds to step ST58.

In step ST57, the image processing apparatus determines that the material of the material determination target is an unknown material, and the processing proceeds to step ST58.

In step ST58, the image processing apparatus determines whether determination of all pixels has been completed. The processing of the image processing apparatus 30 proceeds to step ST59 in a case where determination of all pixels has not been finished, and proceeds to step ST60 in a case where determination of all pixels has been completed.

In step ST59, the image processing apparatus updates the determination target pixel. The image processing apparatus 30 selects a new pixel for which determination of the material has not been performed as the determination target pixel, and the processing returns to step ST53.

In step ST60, the image processing apparatus outputs a determination result. The image processing apparatus 30, for example, displays image regions for which materials are determined as the same material with the same color, the same luminance, or the like, on the basis of the determination result of the material. Further, the image processing apparatus 30 displays regions for which materials are determined as different materials with different color or luminance.

In this manner, according to the second operation example, similar to the first operation example, it is possible to determine materials of respective subjects included in an imaging range of the determination target imaging unit 321. Further, a difference in material is indicated as a difference in attribute of display, so that it is possible to easily determine the difference in material. Further, in the second operation example, the light source is specified, so that the material can be determined more easily than the first operation example.

### 5-3. Third Operation Example

In the third operation example, description will be provided regarding the case where the image processing apparatus 30 further includes a detection region setting unit configured to set a target subject detection region from a polarized image obtained by capturing an image of the material determination target; and a region detection unit configured to detect a target subject region from the target subject detection region set at the detection region setting unit on the basis of a material determination result at the determination processing unit.

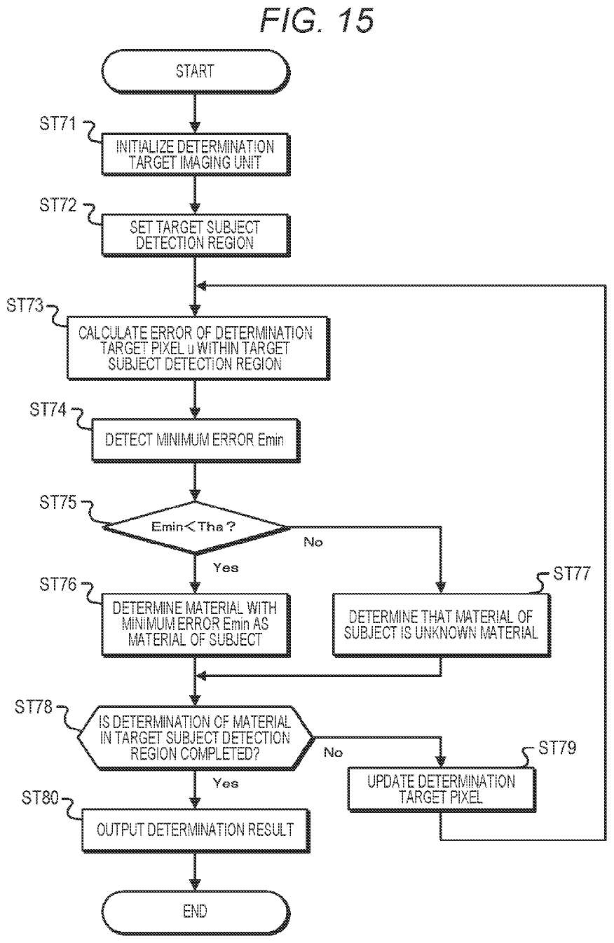

FIG. 15 is a flowchart illustrating a third operation example. In step ST71, the image processing apparatus initializes the determination target imaging unit. The determination target information acquisition unit 32 of the image processing apparatus 30 calibrates the determination target imaging unit 321, and the processing proceeds to step ST72.

In step ST72, a target subject detection region is set. A detection region setting unit of the image processing apparatus 30 sets a target subject detection region including the material determination target (target subject) from the polarized image generated at the determination target imaging unit 321 or a non-polarized image generated on the basis of the polarized image using a method in related art. For example, the detection region setting unit detects a background region and sets a region different from the background region as the target subject detection region, and the processing proceeds to step ST73.

In step ST73, the image processing apparatus calculates an error of a determination target pixel u in the target subject detection region. The determination processing unit 34 of the image processing apparatus 30 calculates an error eij for each combination of an i-th (where i=0, 1, . . . q) type of a material and a j-th (where j=0, 1, . . . , r) type of a light source, and the processing proceeds to step ST74.

In step ST74, the image processing apparatus detects a minimum error Emin. The determination processing unit 34 of the image processing apparatus 30 detects the minimum error Emin in a similar manner to the first operation example, and the processing proceeds to step ST75.

In step ST75, the image processing apparatus determines whether the minimum error Emin is smaller than a threshold Tha. The processing of the determination processing unit 34 of the image processing apparatus 30 proceeds to step ST76 in a case where it is determined that the minimum error Emin detected in step ST74 is smaller than the threshold Tha, and proceeds to step ST77 in a case where the minimum error Emin is equal to or greater than the threshold Tha.

In step ST76, the image processing apparatus determines a material with the minimum error Emin as the material of a subject corresponding to the determination target pixel u, and the processing proceeds to 78.

In step ST77, the image processing apparatus determines that the material of the subject corresponding to the determination target pixel u is an unknown material, and the processing proceeds to step ST78.

In step ST78, the image processing apparatus determines whether determination of the material in the target subject detection region has been completed. The processing of the image processing apparatus 30 proceeds to step ST79 in a case where there remains a pixel for which determination of a material has not been performed in the target subject detection region, and proceeds to step ST80 in a case where determination of materials of respective pixels within the target subject detection region has been completed.

In step ST79, the image processing apparatus updates the determination target pixel. The image processing apparatus 30 selects a new pixel for which determination of the material has not been performed as the determination target pixel, and the processing returns to step ST73.

In step ST80, the image processing apparatus outputs a determination result. The image processing apparatus 30, for example, displays image regions for which materials are determined as the same material with the same color, the same luminance, or the like, on the basis of the determination result of the material. Further, the image processing apparatus 30 displays regions for which materials are determined as different materials with different color or luminance.

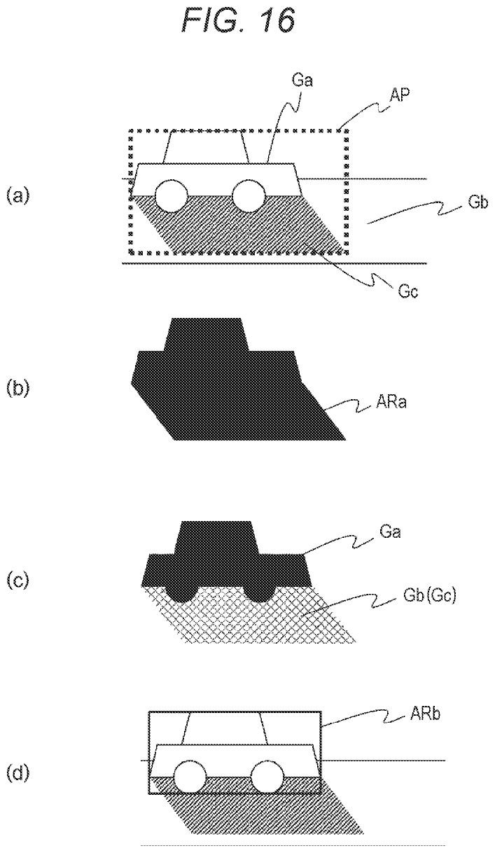

FIG. 16 illustrates the third operation example in which, for example, the target subject is a vehicle Ga, and a road Gb includes shadow Gc of the vehicle Ga. (a) of FIG. 16 indicates an imaging range AP of the determination target imaging unit 321 with a dashed line. (b) of FIG. 16 illustrates a case where a target subject detection region ARa is extracted from an image of the imaging range AP while a background region is excluded using a method in related art, and the target subject detection region ARa includes the shadow Gc of the vehicle generated on the road Gb as well as the vehicle Ga. (c) of FIG. 16 illustrates a material determination result. In a case where a Mueller matrix peculiar to a road is stored in the information storage unit 33, the road Gb in the target subject detection region ARa can be detected. Thus, a region obtained by excluding a portion of the shadow Gc of the road Gb from the target subject detection region ARa can be determined as an image region of the vehicle which is the target subject. Thus, as illustrated in (d) of FIG. 16, for example, a rectangular region ARb indicating the vehicle can be detected with high accuracy.

In this manner, according to the present technology, even in a case where it is difficult to determine a region of a desired subject on the basis of color, luminance, or the like, of the subject, it is possible to segment materials on the basis of material polarizing characteristic information indicating polarizing and reflecting characteristics peculiar to materials which are independent of an external environment, so that it is possible to determine the region of the desired subject.

### 5-4. Other Operation Examples

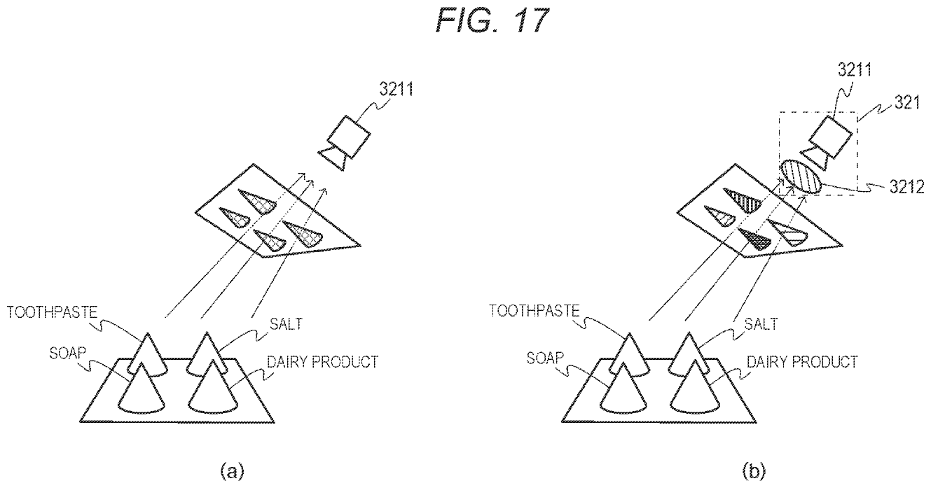

In other operation examples, objects which have substantially the same appearance are distinguished from each other. FIG. 17 illustrates an example of a case where, for example, soap, toothpaste, salt and a dairy product are used as objects which have substantially the same appearance. In this case, material polarizing characteristic information of respective objects generated at the information generation apparatus 20 is stored in the information storage unit 33 in advance. Further, incident polarized light information when an image of the material determination target is captured is stored in the information storage unit 33 in advance.

The image processing apparatus 30 captures an image of the material determination target after calibration and determines a material for which an error from an estimation result using the Mueller matrix of each material as described above is a minimum, as the material of the material determination target. Thus, while objects which have substantially the same appearance cannot be distinguished from each other from an image captured with the imaging unit 3211 in related art as illustrated in (a) of FIG. 17, use of the present technology enables objects which have substantially the same appearance to be distinguished from each other from a difference in materials on the basis of the polarized image acquired with the determination target imaging unit 321 including the imaging unit 3211 and the polarizing plate 3212 as illustrated in (b) of FIG. 17. Further, although not illustrated, objects formed with substantially the same material can be distinguished from each other even in a case where appearance is different due to influence of shadow, or the like.

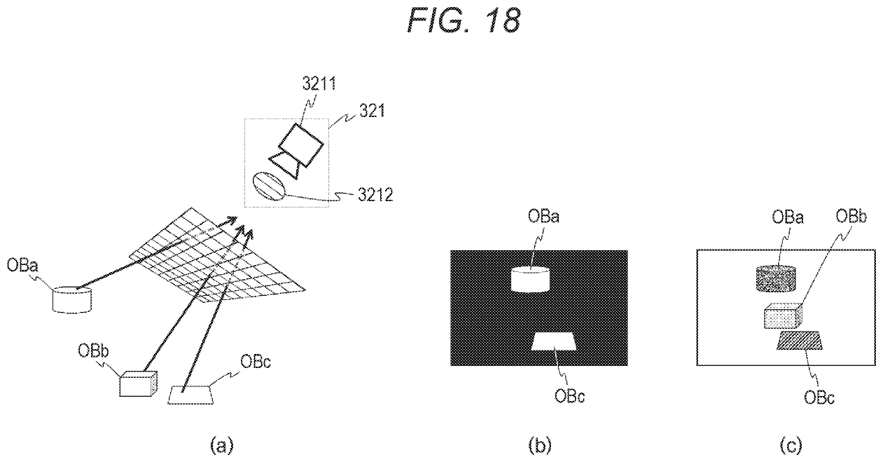

Further, the image processing apparatus 30 can determine a material for each pixel of the polarized image acquired at the determination target imaging unit 321, and thus, a determination result may be presented in image unit. FIG. 18 illustrates an example of a case of presenting a determination result in pixel unit. (a) of FIG. 18 illustrates an example of a case where the polarized image acquired at the determination target imaging unit 321 including the imaging unit 3211 and the polarizing plate 3212 includes objects OBa, OBb and OBc. (b) of FIG. 18 illustrates an example of a determination result, and only displays the objects OBa and OBc for which materials are determined. Further, an error is calculated as described above, and thus, as illustrated in (c) of FIG. 18, gradation, or the like, of objects may be set in accordance with the error so as to enable recognition of determination accuracy of the material.

Further, the image processing apparatus may use a light source appropriate for the material in a case where a target for which a material is to be determined is provided indoors. In this case, the information generation apparatus can determine a material with high accuracy by generating material polarizing characteristic information in a case where a light source appropriate for the material is used.

### 6. Other Configurations and Operation

By the way, while an example of a case has been described where the polarizing plate is provided in front of the imaging unit, and a plurality of polarized images with different polarization directions is acquired by performing imaging while rotating the polarizing plate in the above-described information generation apparatus and image processing apparatus, the polarized images may be acquired using other methods.

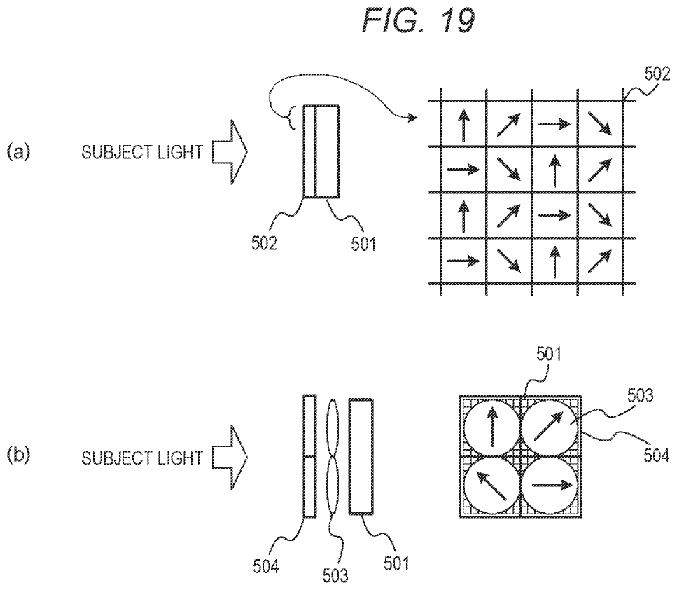

FIG. 19 illustrates an example of other methods for acquiring the polarized images. For example, as illustrated in (a) of FIG. 19, the polarized images are generated by performing imaging in the polarized image acquisition unit where a polarizing filter 502 including a configuration of pixels in a plurality of polarization directions is provided at an image sensor 501. Note that (a) of FIG. 19 illustrates an example of a case where the polarizing filter 502 including pixels in four different polarization directions (polarization directions are indicated with arrows) is disposed in front of the image sensor 501. Further, as illustrated in (b) of FIG. 19, the polarized image acquisition unit may generate a plurality of polarized images in different polarization directions by utilizing a configuration of a multi-lens array. For example, a plurality of (four in the drawing) lenses 503 is provided on a front surface of the image sensor 501, and optical images of the subject are respectively formed on an imaging surface of the image sensor 501 by the respective lenses 503. Further, polarizing plates 504 are provided on front surfaces of the respective lenses 503, and a plurality of polarized images in different polarization directions is generated while setting different directions as polarization directions of the polarizing plates 504. Such a configuration of the polarized image acquisition unit enables a plurality of polarized images to be acquired with one time of imaging. Note that the polarized image acquisition unit may generate polarized images of three primary colors by providing a color filter at the image sensor 501. Further, a plurality of polarized images may be generated by imaging units which are provided for each polarization direction.

### 7. Application Examples

A technology according to the present disclosure can be applied to various fields. For example, the technology according to the present disclosure may also be realized as a device mounted in a mobile body of any type such as automobile, electric vehicle, hybrid electric vehicle, motorcycle, bicycle, personal mobility, airplane, drone, ship, or robot. For example, presentation of an environment around a driver to the driver by utilizing a determination result of the material, makes it easier for the driver to grasp the environment, so that it is possible to reduce fatigue of the driver. Further, it is possible to achieve safer automated driving, or the like. Further, by applying the present technology to equipment, or the like, used in a production process at a factory, it is possible to prevent parts, or the like, of different materials from being mixed in. Still further, by applying the present technology to a surveillance field, it is possible to achieve surveillance operation in view of a material as well as, for example, a shape and movement of an object, by utilizing a determination result of the material.

The series of processes described in the present specification can be executed by hardware, software, or a combination configuration of hardware and software. In a case where the process is executed by software, a program in which a processing sequence is recorded can be installed in a memory in a computer embedded in dedicated hardware to be executed. Alternatively, the program can be installed in a general computer capable of executing various processes to be executed.

For example, the program can be recorded on a hard disk, a solid state drive (SSD) or read only memory (ROM) as a recording medium in advance. Alternatively, the program can be temporarily or permanently stored (recorded) in (on) a removable recording medium such as a flexible disk, a compact disc read only memory (CD-ROM), Magneto Optical (MO) disk, a digital versatile disc (DVD), a Blu-Ray Disc (registered trademark) (BD), a magnetic disk, or a semiconductor memory card. It is possible to provide such a removable recording medium as so-called packaged software.

In addition, the program can be, not only installed on a computer from a removable recording medium, but also transferred wirelessly or by wire to the computer from a download site via a network such as a LAN (Local Area Network) or the Internet. In such a computer, a program transferred in the aforementioned manner can be received and installed on a recording medium such as built-in hardware.

In addition, the effects described in the present specification are not limiting but are merely examples, and there may be additional effects that are not described above. Further, the present technology is not interpreted as being limited to the above-described embodiments of the technology. The embodiments of the technology disclose the present technology in the form of exemplification, and it is obvious that a person skilled in the art can make modification or substitution of the embodiments without departing from the gist of the present technology That is, the gist of the present technology should be determined in consideration of the claims.

Additionally, the image processing apparatus of the present technology may also be configured as below.

(1) An image processing apparatus including:

a determination environment information acquisition unit configured to acquire incident polarized light information of a light source in a material determination environment;

a determination target information acquisition unit configured to acquire emitted polarized light information from a polarized image obtained by capturing an image of a material determination target in the material determination environment; and

a determination processing unit configured to determine a material of the material determination target on the basis of the incident polarized light information acquired at the determination environment information acquisition unit, the emitted polarized light information acquired at the determination target information acquisition unit and material polarizing characteristic information which indicates polarizing and reflecting characteristics for each incident direction of incident polarized light and for each emission direction of reflected light, and which is generated in advance.

(2) The image processing apparatus according to (1), in which the determination processing unit calculates an error of one of the incident polarized light information and the emitted polarized light information estimated using material polarizing characteristic information selected in accordance with an incident direction of incident polarized light on the material determination target and an emission direction of reflected light from the material determination target and the other of the incident polarized light information acquired at the determination environment information acquisition unit and the emitted polarized light information acquired at the determination target information acquisition unit, and determines the material of the material determination target on the basis of the calculated error.

(3) The image processing apparatus according to (2), in which the determination processing unit generates estimated emitted polarized light information using the selected material polarizing characteristic information and the incident polarized light information and determines the material of the material determination target on the basis of an error between the estimated emitted polarized light information and the emitted polarized light information acquired at the determination target information acquisition unit.

(4) The image processing apparatus according to (2), in which the determination processing unit calculates estimated incident polarized light information using the selected material polarizing characteristic information and the emitted polarized light information acquired at the determination target information acquisition unit and determines the material of the material determination target on the basis of an error between the estimated incident polarized light information and the incident polarized light information acquired at the determination environment information acquisition unit.

(5) The image processing apparatus according to any one of (2) to (4), in which the material polarizing characteristic information is generated for each of a plurality of materials; and

the determination processing unit selects, according to respective materials, material polarizing characteristic information corresponding to an incident direction of the incident polarized light and an emission direction of reflected light from the material polarizing characteristic information, calculates the error for each material and determines a material for which the error is a minimum as the material of the material determination target.

(6) The image processing apparatus according to (5), in which the material polarizing characteristic information is generated for each of a plurality of materials, and

in a case where a minimum error in the calculated errors is smaller than a threshold set in advance, the determination processing unit determines a material with the minimum error as the material of the material determination target.

(7) The image processing apparatus according to any one of (2) to (6), in which the determination environment information acquisition unit acquires the incident polarized light information for each of a plurality of light sources in the material determination environment, and

the determination processing unit calculates the error using the incident polarized light information for each of the plurality of light sources and determines a material with a minimum error as the material of the material determination target.

(8) The image processing apparatus according to any one of (2) to (6), in which the determination environment information acquisition unit acquires the incident polarized light information for each of a plurality of light sources in the material determination environment, and

the determination processing unit calculates the error using incident polarized light information of a light source selected from the incident polarized light information for each of the plurality of light sources and determines a material with a minimum error as the material of the material determination target.

(9) The image processing apparatus according to any one of (1) to (6), further including: a detection region setting unit configured to set a target subject detection region from a polarized image obtained by capturing an image of the material determination target; and

a region detection unit configured to detect a target subject region from the target subject detection region set at the detection region setting unit on the basis of a material determination result at the determination processing unit.

(10) The image processing apparatus according to any one of (1) to (9), in which the incident polarized light information acquired at the determination environment information acquisition unit, and the material polarizing characteristic information are stored in an information storage unit in advance, and the determination processing unit determines the material of the material determination target using the incident polarized light information and the material polarizing characteristic information stored in the information storage unit.

(11) The image processing apparatus according to any one of (1) to (10), in which the determination environment information acquisition unit segments the material determination environment into a plurality of regions and sets an average incident direction and average incident polarized light information for each region as an incident direction and incident polarized light information of the region.

(12) The image processing apparatus according to any one of (1) to (11), in which the determination processing unit determines the material of the material determination target on the basis of normalized incident polarized light information, emitted polarized light information and material polarizing characteristic information.

Additionally, the information generation apparatus of the present technology may also be configured as below.

(1) An information generation apparatus including:

a light source information acquisition unit configured to acquire incident polarized light information of incident polarized light on an information generation target from a light source in a measurement environment in which the information generation target whose material is obvious is provided, for each incident direction;

an emitted polarized light information acquisition unit configured to acquire emitted polarized light information of reflected light from the information generation target for each emission direction; and

a material polarizing characteristic information generation unit configured to generate material polarizing characteristic information which indicates polarizing and reflecting characteristics in an incident direction of the incident polarized light and in an emission direction of the reflected light for each direction using the incident polarized light information acquired at the light source information acquisition unit and the emitted polarized light information acquired at the emitted polarized light information acquisition unit.

(2) The information generation apparatus according to (1), in which a plurality of the materials is provided,

the light source information acquisition unit acquires the incident polarized light information for each material,

the emitted polarized light information acquisition unit acquires the emitted polarized light information for each material, and

the material polarizing characteristic information generation unit generates material polarizing characteristic information indicating polarizing and reflecting characteristics for each incident direction and for each emission direction, for each material.

(3) The information generation apparatus according to (1) or (2), in which the emitted polarized light information acquisition unit acquires the emitted polarized light information on the basis of an information generation target imaging unit configured to generate polarized images in a plurality of polarization directions by capturing an image of the information generation target and observation values of polarized images generated at the information generation target imaging unit.

(4) The information generation apparatus according to any one of (1) to (3), in which the material polarizing characteristic information generation unit generates normalized material polarizing characteristic information.

(5) The information generation apparatus according to any one of (1) to (4), in which the incident polarized light information indicates a Stokes vector of the incident polarized light, the emitted polarized light information indicates a Stokes vector of the reflected light, and the material polarizing characteristic information indicates a Mueller matrix.

### REFERENCE SIGNS LIST

10 Material determination system
20 Information generation apparatus
21 Light source information acquisition unit
22 Emitted polarized light information acquisition unit
23 Material polarizing characteristic information generation unit
30 Image processing apparatus
31 Determination environment information acquisition unit
32 Determination target information acquisition unit
33 Information storage unit
34 Determination processing unit
50 Database unit
211 Light source imaging unit
212 Incident polarized light information calculation unit
221 Known material imaging unit
222 Emitted polarized light information calculation unit
231 Polarizing and reflecting characteristic calculation unit
232 Material polarizing characteristic information generation unit
311 Environment imaging unit
312 Incident polarized light information calculation unit
321 Determination target imaging unit
322 Emitted polarized light information calculation unit
341 Estimation processing unit
342 Error calculation unit
343 Material determination processing unit
501 Image sensor
502 Polarizing filter
503 Lens
504 Polarizing plate
3111, 3211 Imaging unit
3112, 3212 Polarizing plate

1. An image processing apparatus comprising:
a determination environment information acquisition unit configured to acquire incident polarized light information of a light source in a material determination environment;
a determination target information acquisition unit configured to acquire emitted polarized light information from a polarized image obtained by capturing an image of a material determination target in the material determination environment; and
a determination processing unit configured to determine a material of the material determination target on a basis of the incident polarized light information acquired at the determination environment information acquisition unit, the emitted polarized light information acquired at the determination target information acquisition unit and material polarizing characteristic information which indicates polarizing and reflecting characteristics for each incident direction of incident polarized light and for each emission direction of reflected light, and which is generated in advance.

2. The image processing apparatus according to claim 1,
wherein the determination processing unit calculates an error of one of the incident polarized light information and the emitted polarized light information estimated using material polarizing characteristic information selected in accordance with an incident direction of incident polarized light on the material determination target and an emission direction of reflected light from the material determination target and the other of the incident polarized light information acquired at the determination environment information acquisition unit and the emitted polarized light information acquired at the determination target information acquisition unit, and determines the material of the material determination target on a basis of the calculated error.

3. The image processing apparatus according to claim 2,
wherein the determination processing unit generates estimated emitted polarized light information using the selected material polarizing characteristic information and the incident polarized light information and determines the material of the material determination target on a basis of an error between the estimated emitted polarized light information and the emitted polarized light information acquired at the determination target information acquisition unit.

4. The image processing apparatus according to claim 2,
wherein the determination processing unit calculates estimated incident polarized light information using the selected material polarizing characteristic information and the emitted polarized light information acquired at the determination target information acquisition unit and determines the material of the material determination target on a basis of an error between the estimated incident polarized light information and the incident polarized light information acquired at the determination environment information acquisition unit.

5. The image processing apparatus according to claim 2,
wherein the material polarizing characteristic information is generated for each of a plurality of materials, and
the determination processing unit selects, according to respective materials, material polarizing characteristic information corresponding to an incident direction of the incident polarized light and an emission direction of reflected light from the material polarizing characteristic information, calculates the error for each material and determines a material for which the error is a minimum as the material of the material determination target.

6. The image processing apparatus according to claim 5,
wherein the material polarizing characteristic information is generated for each of a plurality of materials, and
in a case where a minimum error in the calculated errors is smaller than a threshold set in advance, the determination processing unit determines a material with the minimum error as the material of the material determination target.

7. The image processing apparatus according to claim 2,
wherein the determination environment information acquisition unit acquires the incident polarized light information for each of a plurality of light sources in the material determination environment, and
the determination processing unit calculates the error using the incident polarized light information for each of the plurality of light sources and determines a material for which the error is a minimum as the material of the material determination target.

8. The image processing apparatus according to claim 2,
wherein the determination environment information acquisition unit acquires the incident polarized light information for each of a plurality of light sources in the material determination environment, and
the determination processing unit calculates the error using incident polarized light information of a light source selected from the incident polarized light information for each of the plurality of light sources and determines a material for the error is a minimum as the material of the material determination target.

9. The image processing apparatus according to claim 1, further comprising:
a detection region setting unit configured to set a target subject detection region from a polarized image obtained by capturing an image of the material determination target; and
a region detection unit configured to detect a target subject region from the target subject detection region set at the detection region setting unit on a basis of a material determination result at the determination processing unit.

10. The image processing apparatus according to claim 1,
wherein the incident polarized light information acquired at the determination environment information acquisition unit, and the material polarizing characteristic information are stored in an information storage unit in advance, and the determination processing unit determines the material of the material determination target using the incident polarized light information and the material polarizing characteristic information stored in the information storage unit.

11. The image processing apparatus according to claim 1,
wherein the determination environment information acquisition unit segments the material determination environment into a plurality of regions and sets an average incident direction and average incident polarized light information for each region as an incident direction and incident polarized light information of the region.

12. The image processing apparatus according to claim 1,
wherein the determination processing unit determines the material of the material determination target on a basis of normalized incident polarized light information, emitted polarized light information and material polarizing characteristic information.

13. An image processing method comprising:
acquiring incident polarized light information of a light source in a material determination environment at a determination environment information acquisition unit;
acquiring emitted polarized light information from a polarized image obtained by capturing an image of a material determination target in the material determination environment at a determination target information acquisition unit; and
determining a material of the material determination target at a determination processing unit on a basis of the incident polarized light information acquired at the determination environment information acquisition unit, the emitted polarized light information acquired at the determination target information acquisition unit and material polarizing characteristic information which indicates, for each material, polarizing and reflecting characteristics for each incident direction of incident polarized light and for each emission direction of reflected light, and which is generated in advance.

14. An information generation apparatus comprising:
a light source information acquisition unit configured to acquire incident polarized light information of incident polarized light on an information generation target from a light source in a measurement environment in which the information generation target whose material is obvious is provided, for each incident direction;
an emitted polarized light information acquisition unit configured to acquire emitted polarized light information of reflected light from the information generation target for each emission direction; and
a material polarizing characteristic information generation unit configured to generate material polarizing characteristic information which indicates polarizing and reflecting characteristics in an incident direction of the incident polarized light and in an emission direction of the reflected light for each direction using the incident polarized light information acquired at the light source information acquisition unit and the emitted polarized light information acquired at the emitted polarized light information acquisition unit.

15. The information generation apparatus according to claim 14,
wherein a plurality of the materials is provided,
the light source information acquisition unit acquires the incident polarized light information for each material,
the emitted polarized light information acquisition unit acquires the emitted polarized light information for each material, and
the material polarizing characteristic information generation unit generates material polarizing characteristic information indicating polarizing and reflecting characteristics for each incident direction and for each emission direction, for each material.

16. The information generation apparatus according to claim 14,
wherein the emitted polarized light information acquisition unit acquires the emitted polarized light information on a basis of an information generation target imaging unit configured to generate polarized images in a plurality of polarization directions by capturing an image of the information generation target and observation values of polarized images generated at the information generation target imaging unit.

17. The information generation apparatus according to claim 14,
wherein the material polarizing characteristic information generation unit generates normalized material polarizing characteristic information.

18. The information generation apparatus according to claim 14,
wherein the incident polarized light information indicates a Stokes vector of the incident polarized light, the emitted polarized light information indicates a Stokes vector of the reflected light, and the material polarizing characteristic information indicates a Mueller matrix.

19. An information generation method comprising:
acquiring incident polarized light information of incident polarized light on an information generation target from a light source in a measurement environment in which the information generation target whose material is obvious is provided, for each incident direction at a light source information acquisition unit;
acquiring emitted polarized light information of reflected light from the information generation target for each emission direction at an emitted polarized light information acquisition unit; and
generating material polarizing characteristic information which indicates polarizing and reflecting characteristics in an incident direction of the incident polarized light and in an emission direction of the reflected light at a material polarizing characteristic information generation unit for each direction using the incident polarized light information acquired at the light source information acquisition unit and the emitted polarized light information acquired at the emitted polarized light information acquisition unit.
